(19) United States
(12) Patent Application Publication
(10) Pub. No.: US 2025/0154456 A1
(43) Pub. Date: May 15, 2025

(54) NEURAL PROGENITOR CELL COMPOSITIONS AND METHODS OF USING THE SAME

(71) Applicant: The General Hospital Corporation, Boston, MA (US)

(72) Inventors: Allan M. GOLDSTEIN, Newton, MA (US); Rhian Gordon STAVELY, Beverly, MA (US); Ryo HOTTA, Weston, MA (US)

(21) Appl. No.: 18/838,347

(22) PCT Filed: Feb. 14, 2023
(86) PCT No.: PCT/US2023/062581

§ 371 Date: Aug. 14, 2024

Related U.S. Application Data
(60) Provisional application No. 63/310,365, filed on Feb. 15, 2022.

(51) Int. Cl. C12N 5/0797; A61K 35/30; A61P 25/00

(52) U.S. Cl. CPC C12N 5/0623; A61K 35/30; A61P 25/00; C12N 2500/30; C12N 2500/84; C12N 2501/734; C12N 2506/13; C12N 2513/00; C12N 2533/52

(57) ABSTRACT

The claimed invention is directed to compositions including adipose tissue-derived, for example, subcutaneous adipose tissue-derived or visceral adipose tissue-derived neuronal progenitor cells, and methods for the production and uses thereof.

### PRIORITY

This application claims benefit of the filing date of U.S. provisional application No. 63/310,365, filed Feb. 15, 2022, the content of which is hereby incorporated by reference in its entirety.

### BACKGROUND OF THE INVENTION

The adipose tissue, e.g., subcutaneous adipose tissue (SAT), contains a reservoir of adipose stem cells that can be readily obtained, such as by minimally-invasive liposuction procedures (Zuk et al., 2002). These cells have been examined in over 270 clinical trials that support favorable patient safety profiles (Chu et al., 2019). Adipose stem cells also contain a neural stem cell (NSC) population that offer a potential treatment option for neurological diseases (Peng, Lu, Li, & Hu, 2019). These cells are understood to arise from mesenchymal stem cells (MSCs) transdifferentiated to a neurogenic lineage, although other theories have been postulated (Krabbe, Zimmer, & Meyer, 2005; Phinney & Prockop, 2007). Regardless, adipose tissue-derived NSCs, e.g., SAT-derived NSCs (SAT-NSCs) comprise only a small population of adipose stem cells with an unknown physiological niche. Due to these confines, there are a lack of protocols to isolate, expand and purify SAT-NSCs in order to examine their properties and evaluate their therapeutic utility as a cellular therapy in preclinical models, despite almost two decades of research.

There continues to be a need for the restoration of neuronal deficiencies.

### SUMMARY OF THE INVENTION

Other features and advantages of the invention will be apparent from the Detailed Description, and from the claims. Thus, other aspects of the invention are described in the following disclosure and are within the ambit of the invention.

In one aspect, the invention provides a method of producing human neuronal progenitor cells from adipose tissue, said method comprising the steps of: a) dissociating the adipose tissue to collect nerve fibre bundles (NFBs); and b) culturing NFBs; thereby producing human neuronal progenitor cells from adipose tissue.

In one embodiment, NFBs are cultured until spheroids are formed.

In some embodiments, the NFBs are collected by counter-filtration.

In some embodiments, the nerve NFBs are visualized using a dye.

In some embodiments, the dye is fluoromyelin red.

In another embodiment, the method further comprises the step of expanding the spheroids in human neuroproliferation medium.

In some embodiments, the NFBs are cultured on a fibronectin coated substrate in the presence of Fetal Bovine Serum.

In yet another embodiment, dissociating the adipose tissue comprises i) digesting the adipose tissue; and ii) filtering the digested adipose tissue to separate lipids from NFBs.

In yet another embodiment, digesting the adipose tissue is conducted by contacting the adipose tissue with one or more enzymes.

In yet another embodiment, the enzymes include a dispase and a collagenase. In some embodiments, the collagenase is selected from collagenase type I, collagenase type IA, collagenase type II, collagenase type III, collagenase type IV, collagenase type XI, collagenase type A, collagenase type B, collagenase type C, collagenase type F, collagenase A, collagenase B, collagenase D, collagenase H, collagenase I, collagenase II, collagenase P, collagenase/dispase, liberase, matrix metallopeptidase-1/fibroblast collagenase/interstitial collagenase (MMP-1), MMP-2, MMP-8/neutrophil collagenase, MMP-9, MMP-13/collagenase 3, or MMP-18/collagenase 4. In some embodiments, the collagenase is collagenase type XI. In some embodiments, the collagenase is liberase which contains collagenases.

In some embodiments, the adipose tissue is subcutaneous adipose tissue (SAT).

In some embodiments, the adipose tissue is visceral adipose tissue (VAT).

In some embodiments the human neuronal progenitor cells express Plp1, P75 and SOX10.

In some embodiments the human neuronal progenitor cells express P75 and CD49f.

In another aspect, the invention provides a human neuronal progenitor cell or a population of cells comprising a human neuronal progenitor cell, wherein said human neuronal progenitor cell expresses Plp1, P75 and SOX10.

In some embodiments, the invention provides a human neuronal progenitor cell or a population of cells comprising a human neuronal progenitor cell, wherein said human neuronal progenitor cell expresses P75 and CD49f.

In one embodiment, the human neuronal progenitor cell does not express Zic1 or does not express a significant amount of Zic1.

In yet another aspect, the invention provides a method of producing neuronal function in the colorectum or stomach of a subject in need thereof, said method comprising contacting the smooth muscle wall of the colorectum or stomach with a human neuronal progenitor cell or a population of cells comprising a human neuronal progenitor cell, wherein said human neuronal progenitor cell engrafts into the colorectum or stomach and produces neuronal function.

In one embodiment, the subject suffers from Hirschsprung disease.

In another embodiment, the subject suffers from gastroparesis.

In some embodiments the human neuronal progenitor cells express Plp1, P75 and SOX10.

In some embodiments the human neuronal progenitor cells express P75 and CD49f.

In some embodiments, the invention provides a method of producing neuronal function in the nervous system of a subject in need thereof, said method comprising contacting nerve tissue with a human neuronal progenitor cell or a population of cells comprising a human neuronal progenitor cell, wherein said human neuronal progenitor cell produces neuronal function in the nerve tissue.

In some embodiments, the nerve tissue is damaged.

In some embodiments, the nerve tissue is in the peripheral nervous system.

In some embodiments, the nerve tissue is in the central nervous system.

In some embodiments, the nerve tissue is in the enteric nervous system.

In some embodiments the human neuronal progenitor cells express Plp1, P75 and SOX10.

In some embodiments the human neuronal progenitor cells express P75 and CD49f.

### BRIEF DESCRIPTION OF THE DRAWINGS

The following brief description of the drawings, given by way of example, but not intended to limit the invention to specific embodiments described, may be understood in conjunction with the accompanying figures, incorporated herein by reference.

FIG. 1 is a series of images and graphs depicting characterization of nerve fibres and Schwann cells in the mouse subcutaneous adipose tissue (SAT). Representative low (Panel A) and high (Panel B) magnification images of wholemount preparations of SAT from Plp1^{GFP}; Wnt1-tdT mice. Scale bars=1 mm (Panel A), 200 μm (Panel B) and 100 μm (Panel Bi-Panel Biii). Panel C) Representative dot plots of Wnt1-tdT and Plp1^{GFP} expression of cells from digested SAT. Panel C′) Quantification of the percentage of Plp1 expressing cells within Wnt1⁺ and Wnt1⁻ populations. n=3 mice per group, Unpaired t-test, ***p<0.001. Panel D) Oil red O staining of lipids in cross sections of the SAT from Wnt-tdT mice. Open arrows indicate nerve fibre bundles (NFBs), and closed arrows denote NFB penetrating into the SAT. Scale bar=100 μm. Representative images from Tau^{GFP}; Wnt1-tdT mice of wholemount SAT with penetrating blood vessels (yellow arrow) (Panel E) and cross sections of NFBs (Panel F). Scale bars=500 μm (Panel E) and 50 μm (Panel F). Representative images of NFBs (Panel G) and individual nerve fibre processes (Panel H) in cross sections of SAT from Plp1^{GFP}; BAF53b-tdT mice. Scale bar=50 μm.

FIG. 2 is a series of images and graphs depicting SAT-Neural Stem Cells (SAT-NSCs) originating from the NFBs of SAT in mice. Representative images of digested cells from the SAT of Wnt1-tdT reporter mice with Wnt1⁺ cells displaying spherical (Panel A) and bipolar morphology (Panel A′). Scale bar=50 μm. NFBs from Wnt1-tdT mice visualized in wholemount SAT (Panel B) and after enzymatic digestion of the SAT on a 40 μm cell strainer (Panel B′). Scale bar=1 mm. Panel C) Percentage of Wnt1⁺ cells within filtered digested SAT (SAT) and cultured SAT-derived spheroids from filtered SAT without NFBs (NFB⁻) and unfiltered SAT containing NFBs (NFB⁺). SAT, n=4; NFB⁻, n=5 and NFB⁺, n=6 mice per group, One-way ANOVA with Holm-Šídák's multiple comparisons test, ***p<0.001. Representative images of heterogenous SAT-derived spheroids (Mixed) (Panel D) and spheroids cultured from purified Wnt1⁺ cells (Purified) (Panel D′) in free floating culture conditions. Scale bars=200 μm (Panel D) and 200 μm (Panel D′). Panel E) and E′) Wnt1-tdT expression in cross sections of mixed SAT-derived spheroids (Panel E) and spheroids generated from purified Wnt1⁺ cells (Panel E′). Scale bar=200 μm. Panel F) Representative images of adipogenesis assay using Wnt1⁻ cells (top row) and Wnt1⁺ cells (bottom row) cultured in control or adipogenesis induction medium and stained for lipid vacuoles with Oil red O. Scale bar=200 μm. Panel G) Quantitative analysis of Oil Red O positive adipocytes from Wnt1⁺ or Wnt1⁻ cells cultured in adipogenesis medium (AM). One-way ANOVA with Holm-Šídák's multiple comparisons test, ****p<0.0001, *p<0.05. Panel H) Quantification of the percentage of Plp1 expressing cells within Wnt1⁺ and Wnt1-populations by flow cytometry. n=3 mice per group, Unpaired t-test, ****p<0.0001. Representative images of adipogenesis assay conducted using heterogenous cells from mixed SAT spheroids expressing Wnt1-tdT (Panel I) Plp1^{GFP} (Panel I′) and bright field images of adipocytes (Panel I″, white arrows). Scale bar=200 μm. Expression of Wnt1-tdT (Panel J) and TUBB3 (Panel J′) in heterogenous cultures from the adipose after neural differentiation. Scale bar=200 μm. Images of Wnt1-tdT expression (Panel K) with immunohistochemistry for GFAP and TUBB3 (Panel K′) and merged images after differentiation. Arrows indicate TUBB3⁺ neurons (open white arrow), GFAP⁺ glial cells (yellow closed arrow) and cells expressing neither marker (white closed arrow). Scale bar=200 μm. Images of BAF53b-tdT and Plp1^{GFP} expression in isolated NFBs from SAT (Panel L) and SAT-NSC neurospheres merged with brightfield (BF) (Panel M). Scale bars=200 μm (Panel L) and 100 μm (Panel M). Panel N) Induction of BAF53b expression from Plp1^{GFP} cells purified from SAT-derived spheroids. Scale bar=250 μm.

FIG. 3 is a series of images depicting Schwann cells contaminating cultures of mesenchymal stem cells (MSCs). Representative images of neural crest-derived (Wnt1-tdT) Schwann cells (Plp1-GFP) in cultures using protocols for MSC isolation from mice. Cells were isolated from digested SAT, filtered for cells<40 μm and were cultured on plastic in monolayer conditions in MSC proliferation medium containing 16.5% FBS, 1% Glutamax and 1% penicillin-streptomycin in α-MEM basal media.

FIG. 4 is a graph and a series of images depicting Plp1 expressing SAT-NSCs differentiating to neurons in vitro. Panel A) Representative flow cytometry plot of cells isolated from the SAT and cultured in free-floating conditions in neuroproliferation medium from Plp1-GFP; BAF53B-tdT transgenic mice. Panel B) Validation by microscopy of transgene expression profiles in Plp1⁺, BAF53B⁺ and double positive cells from SAT-derived spheroids isolated by flow cytometry and cultured on fibronectin. Panel C) Identification of Plp1⁺, BAF53B⁺ and double positive cells in cultures of only Plp1⁺ cells purified by flow cytometry indicating Schwann-like cells gives rise to BAF53B⁺ neurons in vitro. Panel D-Panel E) Representative images of Plp1⁺ cell differentiation into multipolar BAF53B⁺ Plp1-neurons in 3D culture conditions. Panel F) No evidence of BAF53B⁺ neuron differentiation was observed in cultures of Plp1-BAF53B-negative cells in the same culture conditions.

FIG. 5 is a series of graphs, charts, and images depicting neural crest derived-Schwann cells from SAT acquiring NSC properties in vitro. Over-representation analysis of cellular components (Panel A) and biological processes (Panel A′) for genes upregulated in neurospheres generated from Wnt1⁺ cells compared to Wnt1-tdT⁺ cells obtained from primary SAT. Panel B-Panel B″) Heatmap representation of Schwann cell markers (Panel B), embryonic morphogenesis (Panel B′), as well as (Panel B″) curated Schwann cell, neural crest stem cell and neuronal genes (top to bottom) in Wnt1-tdT⁺ cells from SAT and cultured neurospheres (SAT-NSCs) visualized as Log CPM values. Panel C) Venn diagram of upregulated DEGs common between SAT-NSCs and enteric neural stem cells (ENSCs). Panel D-Panel E) Expression of Wnt1-tdT and Nestin^{GFP} in the NFBs (Panel D) and dispersed Wnt1⁺ Schwann cells (Panel E) in the SAT. Scale bars=200 μm. Panel F) NFBs from the digested SAT of Nestin^{GFP}; Wnt1-tdT cultured into spheroids over 10 days in free floating conditions. Scale bars=500 μm. Panel F′) Quantification of the mean fluorescence intensity (MFI) of Nestin^{GFP} in cultured NFBs. Day 0, n=5; day 3, n=8; day 10; n=7 NFBs per group, One-way ANOVA with Holm-Šídák's multiple comparisons test, **p<0.01, ***p<0.001. Panel G) Representative images of Wnt1-tdT expression, immunoreactivity for P75/NGFR and merged imaged with DAPI of heterogenous SAT-derived spheroids. Scale bars=200 μm. Panel G′) Quantification of the percentage of P75 expressing cells in the Wnt1⁺ and Wnt1-populations of SAT-derived spheroids. N=8 spheroids, Unpaired t-test, ****p<0.0001. Representative images of Wnt1-tdT expression and immunohistochemistry for GDNF (Panel H), Notch1 and Ki67 (Panel I) in neurospheres generated from purified Wnt1⁺ cells. Scale bars=200 μm.

FIG. 6 is a series of images and a graph depicting the gut signaling milieu promoting the enteric differentiation of SAT-NSCs. Procedure to access the colorectum (Panel A) via perianal incision (Panel A′) and neurosphere implantation to the exposed gut wall (Panel A″). Scale bar=5 mm. Panel B) Representative image of bright field microscopy and Wnt1-tdT expression of transplanted SAT-NSCs. Scale bar=5 mm. Representative images of transplanted Wnt1⁺ SAT-NSCs at 2 (Panel C), 4 (Panel C′) and 8 weeks (Panel C″) post implantation. Scale bar=1 mm. Panel D) Quantification of the area occupied by transplanted Wnt1⁺ cells over 8 weeks. n=3 mice per group, One-way ANOVA with post-test for linear trend. Panel E) Cross section of the transplanted colon stained with DAPI. Arrow indicated transplanted Wnt1⁺ cells located between the circular muscle (CM) and longitudinal muscle (LM). Scale bar=100 μm. Panel F) High magnification image of transplanted Wnt1⁺ cells immunolabeled with TUBB3 in cross sections. Scale bar=50 μm. Panel G) Images of wholemount preparations of smooth muscle recipient tissues contained transplanted Wnt1⁺ cells and neurons and nerve fibres immunoreactive for TUBB3. Scale bar=500 μm. Panel H) and Panel H′) Wnt1⁺ cells expressed TUBB3 and formed ganglia-like structures (arrows) at the centre of the transplantation site. Scale bar=100 μm. Representative images of transplanted Wnt1⁺ cells immunolabeled for neuron specific enolase (NSE) (Panel I, Scale bar=50 μm) and expression of Tau^{GFP} (Panel J, Scale bar=500 μm) in wholemount preparations. Panel K), Panel K′), and Panel K″) Representative image of the integration between transplanted Wnt1⁺ cells and the endogenous enteric ganglia (arrows) expressing TUBB3. Scale bar=50 μm. Panel L), Panel L′), Panel L″), and Panel L′″) Immunohistochemical labelling of calretinin (Calr) in wholemount preparations of the colon (Scale bar=500 μm). Panel M-Panel O) Immunohistochemical labelling of neurochemical coding markers for calretinin, (Panel M) cholinergic (VAChT) (Panel N-Panel N″) and neuropeptide Y (NPY) (Panel O) expressing neuronal subtypes in cross sections of the colon. Scale bar=50 μm. Panel P) Visualisation of transplanted Wnt1⁺ cells and immunohistochemical labelling of neuronal NOS (nNOS). Scale bar=50 μm.

FIG. 7 is a series of images depicting SAT-NSCs integrating with the ENS ex vivo and in vitro. Representative images of transplanted cells located alone (Panel A-Panel A″) and in proximity to endogenous myenteric ganglia (Panel B-Panel B″). White arrows indicate endogenous TUBB3⁺ cells. Recipient muscularis propria tissues visualised for Wnt1⁺ cells (Panel A-Panel B), immunohistochemical labelling of TUBB3 (Panel A′-Panel B′) and merged images (Panel A″-Panel B″). Scale bar=50 μm. Panel C-Panel C″) Representative images of co-cultured heterogenous neurospheres isolated from the SAT of Wnt1-tdT mice (SAT-NSCs) and the colonic muscularis propria of Plp1^{GFP} mice (enteric glial cells) on fibronectin. SAT-NSCs and enteric glia migrated out of spheroids (Panel C, Scale bar=500 μm) and began forming connections between each other after one week (Panel C′, Scale bar=200 μm). By 3 weeks, SAT-NSCs exhibited multipolar morphologies (Panel C″, Scale bar=100 μm) with nerve fibre projections penetrating through enteric ganglia-like structures (Panel C″, Scale bar=100 μm).

FIG. 8 is a series of images and graphs depicting transplantation of SAT-NSCs alleviating gastroparesis in nNOS KO mice. Panel A) Implantation of SAT-NSCs proximal to the pylorus in the stomach of the nNOS^{-/-} mouse model of gastroparesis. Panel B) Engraftment of Wnt1-tdT⁺ SAT-NSCs (arrows) into the stomach. Scale bar=2 mm. Panel C) SAT-NSCs with projecting nerve fibres (arrow heads) in the stomach. Scale bar=1 mm. Panel D) Representative images of stomachs from nNOS^{-/-} receiving implantation of SAT-NSCs and sham surgery and implantation controls. Scale bar=1 cm. Panel D′) Quantification of stomach sizes as area in mm². One-way ANOVA with Holm-Šídák's multiple comparisons test, *p<0.05, **p<0.01, ***p<0.001. Naïve wildtype mice, n=5; sham nNOS^{-/-}, n=6 and SAT-NSC treated nNOS^{-/-}, n=9 mice group. Panel E) Representative images of radiographic visualization of gavaged radiopaque beads and liquid barium in the gastrointestinal tract of naïve wildtype, nNOS^{-/-}, nNOS^{-/-} mice with sham surgery and nNOS^{-/-} mice with SAT-NSC transplantation (left to right). Panel F) Quantification of gastric emptying of solid materials (beads). Brown-Forsythe ANOVA test with Welch's corrected multiple comparisons t-test, *p<0.05, **p<0.01. Naïve wildtype mice, n=7; naïve nNOS^{-/-} mice, n=8; sham nNOS^{-/-}, n=9 and SAT-NSC treated nNOS, n=8 mice per group. Panel G) Quantification of gastric emptying of liquid materials (barium). Brown-Forsythe ANOVA test with Welch's corrected multiple comparisons t-test, *p<0.05, **p<0.01. Naïve wildtype mice, n=7; naïve nNOS^{-/-} mice, n=8; sham nNOS^{-/-}, n=9 and SAT-NSC treated nNOS^{-/-}, n=8 mice per group. Panel H-Panel I) Representative single confocal slice images of EdU incorporation in proliferating cells (Panel H), the neuronal marker TUBB3 (Panel H′ Tuj1), DAPI (Panel H′ DAPI), Wnt1-tdT (Panel H′ Wnt1) and merged images (Panel H′ Merge & Panel I) in the muscularis of the stomach ex vivo after Wnt1-tdT⁺ SAT-NSC implantation for 7 days. Scale bar=50 μm. Panel J-Panel J″) Quantification of the total area (mm²) covered by migrating Wnt1-tdT⁺ SAT-NSCs (Panel J), percentage of the TUBB3-immunoreactivity (IR) colocalization area with Wnt1-tdT⁺ SAT-NSCs (Panel J′) and the percentage of proliferating EdU⁺ SAT-NSCs (Panel J″) after 7 days in the stomach (left bar in each graph) and colon (right bar in each graph). Unpaired t-test, *p<0.05, **p<0.01, ***p<0.001, n=4 mice per group.

FIG. 9 is a series of images and graphs depicting SAT-NSCs restoring muscle contraction in intestinal aganglionosis. Panel A) Representative images of the nervous system in the colon of Wnt1-tdT; Ednrb KO mice. Scale bar=4 mm. The proximal colon is ganglionated (G, I) with intrinsic neurons, while the aganglionic (AG, II) segment is observed from the mid colon to the rectum and contains only hypertrophic nerve fibres of extrinsic origin. Scale bar=1 mm. Panel B-Panel B′) Expression of Wnt1-tdT in SAT-NSCs two weeks post transplantation to the colorectum of Ednrb^{-/-} with colorectal aganglionosis at P6. Scale bar=0.5 mm. Panel C-Panel C′) Images of wholemount preparations of the Ednrb KO colon with transplanted Wnt1-tdT⁺ SAT-NSCs (Panel C) and immunolabelled for the neuronal marker TUBB3 (Panel C′). Scale bar=300 μm. Panel D) Cross section of the transplanted Ednrb KO colon in bright field. Arrows indicate transplanted Wnt1⁺ cells located between the mucosa and circular muscle (CM) or the CM and longitudinal muscle (LM). Scale bar=200 μm. Panel E) Cross section of the SAT-NSC transplanted Ednrb KO colon labelled for alpha smooth muscle actin (SMA) and stained with DAPI. Scale bar=200 μm. Panel F-Panel F′) Representative images of the muscularis (SMA) in cross sections of the SAT-NSC transplanted Ednrb KO colon labelled with the neuronal marker TUBB3. Scale bar=200 μm. Panel G) Representative traces of colonic contractile force in Ednrb^{+/+} (Ednrb WT, black), Ednrb^{-/-} (Ednrb KO, red) and Ednrb^{-/-} mice with SAT-NSC neurosphere implantation (Ednrb KO+SAT-NSC, green) before (spontaneous) and directly after EFS. Panel H) Quantification of the change in contractile force (grams) from baseline after EFS stimulation in Ednrb WT, Ednrb KO and Edrnb KO mice after SAT-NSC transplants. Ednrb KO, n=6; Ednrb WT and Edrnb KO+SAT-NSC, n=4 mice per group. Kruskal-Wallis nonparametric ANOVA with Dunn's multiple comparisons test, *p<0.05. Panel H′) Quantification of TTX sensitive (neural) contributions to the EFS-induced contractile response. Ednrb WT, n=4; Ednrb KO, n=6 and Edrnb KO+SAT-NSC, n=3 mice per group. Kruskal-Wallis nonparametric ANOVA with Dunn's multiple comparisons test, *p<0.05. Panel I-Panel I′) Representative single confocal slice images of EdU incorporation in proliferating cells (EdU) the neuronal marker TUBB3, DAPI and Wnt1-tdT in the muscularis of the ganglionated Ednrb WT (Panel I) and aganglionic Ednrb KO (Panel I′) distal colon after Wnt1-tdT⁺ SAT-NSC implantation for 7 days. Scale bar=50 μm. Panel J-Panel J″) Quantification of the total area (mm²) covered by migrating Wnt1-tdT⁺ SAT-NSCs (Panel J), percentage of the TUBB3-immunoreactivity (IR) colocalization area with Wnt1-tdT⁺ SAT-NSCs (Panel J′) and the percentage of proliferating EdU⁺ SAT-NSCs (Panel J″) after 7 days in the ganglionated (Ednrb WT) and aganglionic (Ednrb KO) colon. Ednrb WT, n=4 and Ednrb KO, n=3 mice per group, Unpaired t-test, NS=not significant. Panel K) Quantification of the number of spontaneous and EFS-invoked calcium responses in SAT-NSCs transplanted to the aganglionic colon. n=3 per group, Unpaired t-test, *p<0.05. Panel L) Representative traces of calcium transients (ΔF/Fo) in transplanted SAT-NSCs in the aganglionic colon. Red traces (Cell 3 and Cell 4) denote neuronal-like responses to EFS, and blue traces (Cell 1 and Cell 2, highest and second highest peak, respectively) denote glial-like secondary responses. Panel M) Representative traces of global calcium transients (ΔF/Fo) from transplanted SAT-NSCs (solid) and smooth muscle contraction (dotted) in response to EFS in the aganglionic colon.

FIG. 10 is a series of images and charts depicting human adult SAT as a source of SAT-NSCs derived from NFBs. Panel A) Sectional slice of resected human SAT. Scale bar=1 cm. Panel B-Panel B′) Immunohistochemistry of the Schwann cell marker, CDH19 (Panel B) and nerve fibre marker, TUBB3 (Panel B′) in cross sections of the superficial SAT. Scale bar=1 cm. Panel C) Representative image of NFB isolated from digested SAT. Scale bar=2 mm. Panel D) Representative images of bright field TUBB3 immunohistochemistry, DAPI and merged images (left to right) in a wholemount NFB from the SAT. Scale bar=500 μm. Panel E) High magnification image of wholemount NFB containing nucleated cells (DAPI) and expressing TUBB3. Scale bar=50 μm. Panel F-Panel F′) Representative images of human SAT-derived NFB before (Panel F, Scale bar=2 mm) and after culture (Panel F′, Scale bar=1 mm) in free floating conditions. Representative images of immunohistochemical labelling for P75 (Panel G, Scale bar=500 μm) and SOX10 (Panel H, Scale bar=200 μm) in SAT NFB-derived spheroids cultured on fibronectin. Panel I) Heatmap representation of curated neural crest stem cell, neuronal, neural development, neurotrophic and Schwann cell genes in SAT NFB-derived spheroids compared to filtered single cell suspension-derived spheroids visualized as Log CPM values. Panel J) Venn diagram of upregulated genes common between human SAT NFB-derived spheroids compared to single cell suspensions and mouse SAT-NSCs compared to Schwann cells. Panel J′) Heatmap representation of common genes in SAT NFB-derived spheroids compared to filtered single cell suspension-derived spheroids visualized as Log CPM values. Panel K) Representative image of human SAT NFB-derived cells labelled with TUBB3 after ex vivo transplantation. Scale bar=200 μm.

FIG. 11 is a series of images and graphs depicting human SAT-NSC transplantation experiments in the aganglionic colon of Ednrb null mice. Panel A) Representative image of human SAT-NSCs in in vitro monolayer culture expressing GFP after lentiviral transduction. Panel B) Representative image of human SAT-NSCs expressing GFP after transplantation to the muscularis externa of the aganglionic colon from Ednrb null mice in ex vivo cultures. Panel C) Preparation of sheets of the muscularis externa for force transduction measurements along the circumferential axis of the tissue. Panel D) Representative traces of force contraction in response to electric field stimulation (EFS) in samples from wildtype, Ednrb KO, and Ednrb KO mice treated with human SAT-NSCs. A notable lack of smooth muscle contractions in response to EFS and rhythmic EFS independent myogenic contractions are characteristic of aganglionosis in Ednrb KO mice. Panel E) Bar graph representation of contractile responses to EFS in samples from untreated Ednrb KO mice and those treated with human SAT-NSCs. Panel F) Bar graph representation of contractile responses to EFS before and after the addition of TTX to inhibit neural function.

FIG. 12 is a series of images depicting neurosphere isolation from the visceral adipose tissue (VAT) of mice. Panel A-Panel A′) Representative images of the intraabdominal cavity of Plp1-GFP; Baf53b-tdT reporter mice showing the mesenteric fat and small intestine. Note the high expression of Plp1-GFP and Baf53b-tdT expression in the enteric nervous system of the intestine and in nerve fibers in the mesenteric adipose projecting to the intestine. Panel B-Panel B′) High magnification images of the previous demonstrating nerve fibers from the mesenteric adipose directly innervating the small intestine. Panel C-Panel C′) Representative image of the mesenteric adipose tissue harvested from the Plp1-GFP; Baf53b-tdT reporter mouse. Panel D-Panel D′) Isolation of nerve fibers from the mesenteric adipose tissue by enzymatic digestion and counter filtration. Panel E) Representative image of a neurosphere cultured from NFBs of the mesenteric adipose tissue with loss of Baf53b-tdT expression. Panel F-Panel F″) Expression of Plp1-GFP and Baf53b-tdT in mesenteric adipose-derived neurospheres cultured on fibronectin with FBS to induce neural differentiation. Panel G-Panel G″) High magnification images of the above demonstrating the elongated fiber projections of Baf53b-tdT neurons. Panel H-Panel H″) Transplantation of mesenteric adipose-derived neurospheres to explants of the aganglionic colon of Ednrb null mice exhibiting expression of Plp1-GFP and neuronal differentiation signified by expression of Baf53b-tdT and immunoreactivity for Tuj1.

FIG. 13 is a series of images depicting the isolation of NFBs and neurospheres from the visceral adipose tissue. Panel A) Representative image of VAT collected from the omentum. Panel B) Image of an NFB isolated from VAT after enzymatic digestion and counter filtration. Panel B′) High magnification image of the same showing myelinated nerve fibers. Panel C-Panel C″) Fluoromyelin staining of an NFB isolated from the VAT. Panel D) Neurosphere generation from NFBs 10 days after initial isolation and staining with fluoromyelin. Panel E-Panel E″) Representative image of VAT-derived cells expressing adipose neural stem cell markers P75 and CD49f.

FIG. 14 is a series of images and graphs depicting the formation of spheroids enriched for neural stem cells from subcutaneous and visceral sources of adipose tissue. Panel A) Representative images of subcutaneous, epiploic, and omental adipose tissue. Panel B) Representative image of spheroids generated from VAT. Panel C) Representative image of P75 immunoreactivity in adipose cell from spheroids cultured from filtered single cell suspensions and the counter filtered material demonstrating increased numbers of P75⁺ cells in the counter filtered sample. Expression of PLP1 (Panel D) and NGFR (Panel E), in spheroids generated from the counter filtered material compared to the filtered single cell suspension from the same subject indicating enrichment of the neural stem cell population.

FIG. 15 is a series of images and graphs depicting the transplantation of SAT-NSCs to the sciatic nerve in a model of nerve gap injury. Panel A) Representative image of silicone conduit loaded with Wnt1-tdT expressing SAT-NSC neurospheres prior to surgical application. Panel B) Representative image of the surgical procedure after sciatic nerve transection and application of the conduit. Panel C-Panel C″) Representative images of the intact conduit, transplanted SAT-NSCs expressing Wnt1-tdT and Schwann cells from the recipient expressing Plp1-GFP. Panel D) Dissection of the conduit containing the intact sciatic nerve at the proximal and distal ends. Panel E-Panel E″) Representative image of the sciatic nerve after removal of the conduit forming a fully intact nerve. Panel F) Higher magnification image of the previous showing the gradual integration between Wnt1-tdT SAT-NSCs and Plp1-GFP expressing Schwann cells from the recipient. Panel G-Panel G′) Representative images of the cell morphology of SAT-NSCs in the integrating nerve. Panel H) Bar chart representation of the leg circumference index in sham-treated (n=4 mice) and SAT-NSC treated mice (n=3 mice) after sciatic nerve transection. *p<0.05, t-test. Panel I) Representative electromyography traces in the gastrocnemius in response to sciatic nerve stimulation. Gray traces (top) represent unoperated hindlegs with an intact sciatic nerve as an internal control. The blue trace (bottom) represents mice with sciatic nerve transection and placement of the conduit without cell treatment (sham) and the red trace (bottom) represents mice with nerve transection and SAT-NSC treatments.

### DEFINITIONS

Unless otherwise defined, all technical and scientific terms used herein have the same meaning as commonly understood by one of ordinary skill in the art to which this invention belongs. In case of conflict, the present application, including definitions will control.

A “subject” is a vertebrate, including any member of the class Mammalia, including humans, domestic and farm animals, and zoo, sports, or pet animals, such as mouse, rabbit, pig, sheep, goat, cattle, and higher primates.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

By “effective amount” is meant the amount of neuronal progenitor cells that produce the desired therapeutic response (i.e., producing, restoring and/or enhancing neuronal function).

By “neuronal progenitor cell” is meant a multipotent cell which has the potential to become committed to the neuronal lineage.

As used herein, the term “human neuroproliferation medium” refers to a medium that can be used to culture human neural stem cells and progenitor cells from different types of tissues, such as, tumors and normal tissues. This expansion of cells is done in the neurosphere or adherent monolayer system.

Human neuroproliferation medium can include, but is not limited to, basic fibroblast growth factor (20 ng/ml; Stemcell Technologies), epidermal growth factor (20 ng/ml; Stemcell Technologies), heparin (0.0002%; Stemcell Technologies), GlutaMAX (1%; Life Technologies), B27 supplement (1%), Primocin (1%), Metronidazole (50 μg/ml), and FBS (5%) in Dulbecco's modified Eagle's medium: Nutrient Mixture F-12 (Gibco, Life Technologies).

The phrase “produces neuronal function” refers to the function exhibited by engrafted neural progenitor cells of the invention which differentiate and provide one or more neuronal activities. Neuronal differentiation can be determined, for example, by detecting immunoreactivity of the neuronal marker TUBB3 and/or observing formation of ganglia-like structures containing neurons. Neuronal activity can be determined, for example, by monitoring calcium influx following electric field stimulation (EFS). By “isolated” is meant a material that is free to varying degrees from components which normally accompany it in its native state. “Isolate” denotes a degree of separation from original source or surroundings.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “adipose tissue” refers to a type of loose connective tissue that extends throughout the body and is important for the storage of energy in the form of lipids. Adipose tissue is also called body fat. It can be found under the skin, between the internal organs and in the inner cavities of bones.

As used herein, the term “subcutaneous adipose tissue” or “SAT” refers to a type of adipose tissue that is found beneath the skin.

As used herein, the term “visceral adipose tissue” or “VAT” refers to a type of adipose tissue that is found in the lining of the internal organs of the body.

As used herein, the term “nerve tissue” refers to the tissue that forms the nervous system such as the tissue that forms the brain, spinal cord, peripheral nerves etc. It is also called nervous tissue or neural tissue and is made up of neurons which relay information to other neurons via synapses.

As used herein, the term “peripheral nervous system” or “PNS” refers to the nerves in the peripheral nervous system, which includes the nerves outside the brain and spinal cord, for example, the nerves in limbs.

As used herein, the term “central nervous system” or “CNS” refers to the nerves in the central nervous system, which includes the nerves in the brain and spinal cord.

As used herein, the term “enteric nervous system” or “ENS” refers to the nerves that are present in the lining of the gastrointestinal system starting from the esophagus to the anus.

As used herein, the term “damaged nerve tissue” refers to any neural tissue that has been damaged or injured either by accident, stroke, fall, etc. It is also referred to as nerve injury and peripheral nerve damage may be referred to as peripheral neuropathy. Nerve damage negatively affects neuronal communication.

As used herein, the term “nerve injury” refers to any damage to the nerve that is caused pressure, stretching, or cutting. Nerve injury can prevent nerves from sending and receiving signals, e.g., it can stop communication with the brain. It has several symptoms such as pain, numbness, weakness, etc.

As used herein, the term “peripheral nerve injury” or “peripheral nervous system injury” refers to any damage to the nerves in the peripheral nervous system, which includes the nerves outside the brain and spinal cord. This type of damage to the peripheral nerves is also called peripheral neuropathy. This kind of damage can affect different bodily functions such as digestion, urination, circulation, etc.

As used herein, the term “central nervous system injury” refers to any damage to the nerves in the central nervous system, which includes the nerves in the brain and spinal cord. This type of nerve damage can have several causes such as accidents, sports injuries, stroke, fall, ruptured brain aneurysms etc. These injuries are also called traumatic brain injuries or traumatic spine injuries.

As used herein, the term “enteric nervous system injury” refers to any damage to the nerves that are present in the lining of the gastrointestinal system starting from the esophagus to the anus. This type of nerve damage can cause problems with the proper functioning of the gastrointestinal tract. Other definitions appear in context throughout this disclosure.

### DETAILED DESCRIPTION

Embryonic Schwann cell precursors are multipotent stem cells that migrate along embryonic nerve fibres and contribute to non-glial cell populations such as melanocytes, neuroendocrine chromaffin cells, enteric neurons, sympathetic neurons and mesenchymal stem cells (MSCs) from the bone marrow depending on local environmental cues (Kameneva, Kastriti, & Adameyko, 2020). However, no equivalent progenitors are known to exist postnatally in the nerve fibre niche (Furlan & Adameyko, 2018; Kameneva et al., 2020). It is now shown that neural crest (NC)-derived Schwann cells isolated from the adipose tissue, e.g., subcutaneous adipose tissue (SAT) or visceral adipose tissue (VAT), are the only cell population displaying gliogenic and neurogenic potential consistent with adipose tissue-derived neural stem cells (NSCs), e.g., SAT-derived NSCs (SAT-NSCs) or VAT-derived NSCs (VAT-NSCs). These cells are distinct from adipose-derived MSCs and reside within the local nervous system niche of the adipose tissue, e.g., SAT or VAT. Methods for their enrichment from nerve fibre bundles (NFBs) in mice and humans are described. Following transplantation into the gastrointestinal tract, SAT-NSCs and VAT-NSCs are receptive to the gut signaling milieu where they efficiently engraft, migrate, and differentiate into enteric neurons and glia, indicating that SAT-NSCs and VAT-NSCs have unprecedented potential as a source of autologous NSCs for peripheral nervous system disorders and for treating nerve injury. Accordingly, the present invention provides methods of treating neurogastrointestinal disease and/or disorders or symptoms thereof which comprise administering a therapeutically effective amount of a composition comprising Plp1, P75, and SOX10 or P75 and CD49f expressing neural progenitor cells, described herein, to a subject (e.g., a mammal, such as a human). Thus, one embodiment is a method of treating a subject having a disease characterized by reduced neurogastrointestinal function, such as Hirschsprung disease or gastroparesis. The method includes the step of administering to the subject an effective amount of Plp1, P75, and SOX10 or P75 and CD49f expressing neural progenitor cells sufficient to treat a neurogastrointestinal disease or disorder or symptom thereof. Another embodiment is a method of treating a subject having nerve injury (e.g., peripheral nerve injury, central nervous system injury, or enteric nervous system injury). The method includes the step of administering to the subject an effective amount of Plp1, P75, and SOX10 or P75 and CD49f expressing neural progenitor cells sufficient to treat the nerve injury or symptom thereof. Identifying a subject in need of such treatment can be in the judgment of a subject or a health care professional and can be subjective (e.g., opinion) or objective (e.g., measurable by a test or diagnostic method).

Plp1, P75, and SOX10 or P75 and CD49f expressing neural progenitor cells of the invention are administered according to methods known in the art. Such compositions may be administered by any conventional route, including injection or by gradual infusion over time. The administration may, depending on the composition being administered, for example, by intrathymic, pulmonary, intravenous, intraperitoneal, intramuscular, intracavity, subcutaneous, or transdermal routes of administration. Compositions comprising Plp1, P75, and SOX10 or P75 and CD49f expressing neural progenitor cells are administered in “effective amounts”, or the amounts that either alone or together with further doses produce the desired therapeutic response. Administered cells of the invention can be autologous (“self”) or non-autologous (“non-self,” e.g., allogeneic, syngeneic, or xenogeneic).

Plp1, P75, and SOX10 or P75 and CD49f expressing neural progenitor cells of the invention can be combined with pharmaceutical excipients known in the art to enhance preservation and maintenance of the cells prior to administration. In some embodiments, cell compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A method to potentially increase cell survival when introducing the cells into a subject in need thereof is to incorporate cells of interest into a biopolymer or synthetic polymer. Depending on the subject's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein.

Those skilled in the art will recognize that the components of the compositions should be selected to be chemically inert and will not affect the viability or efficacy of the stem cells or their progenitors as described in the present invention. This will present no problem to those skilled in chemical and pharmaceutical principles, or problems can be readily avoided by reference to standard texts or by simple experiments (not involving undue experimentation), from this disclosure and the documents cited herein. One consideration concerning the therapeutic use of cells is the quantity of cells necessary to achieve an optimal effect. Different scenarios may require optimization of the amount of cells injected into a tissue of interest. Thus, the quantity of cells to be administered will vary for the subject being treated. The precise determination of what would be considered an effective dose may be based on factors individual to each patient, including their size, age, sex, weight, and condition of the patient. As few as 100-1000 cells can be administered for certain desired applications among selected patients. Therefore, dosages can be readily ascertained by those skilled in the art from this disclosure and the knowledge in the art. The skilled artisan can readily determine the amount of cells and optional additives, vehicles, and/or carrier to be administered in conjunction with the compositions and methods of the disclosure. Of course, for any composition to be administered to an animal or human, and for any particular method of administration, it is therefore preferred to determine toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation by the skilled artisan and therefore, the time for sequential administrations can be ascertained without undue experimentation. Plp1, P75, and SOX10 or P75 and CD49f expressing neural progenitor cells of the invention are produced from adipose tissue, e.g., SAT or VAT, that has either been enzymatically or mechanically treated (or both) to enable selection of NFBs. NFBs are transformed in culture into spheroids, which are expanded in human neuroproliferation medium prior to sorting Plp1 expressing cells from the spheroids in order to isolate the neuronal progenitor cells.

Human neuronal progenitor cells are obtained from adipose tissue, e.g., SAT or VAT, by either contacting the adipose tissue, e.g., SAT or VAT with dispase and at least one other collagenase to enzymatically digest the tissue and/or mechanically disassociate and filter the adipose tissue, e.g., SAT or VAT, into separate lipids from NFBs. Isolated NFBs are cultured until spheroids are formed and spheroids are expanded in human neuroproliferation medium to produce human neuronal progenitor cells from adipose tissue, e.g., SAT or VAT.

Cell purification and isolation methods are known to those skilled in the art include, but are not limited to, sorting techniques based on cell-surface marker expression, such as fluorescence activated cell sorting (FACS sorting), positive isolation techniques, and negative isolation, magnetic isolation, and combinations thereof. Those skilled in the art can readily determine the percentage of neuronal progenitors in a population of cells using various well-known methods, such as FACS. Neuronal progenitors may comprise a population of cells that have about 10-15%, 15-20%, 20-25%, 25-30%, 35-40%, 45-50%, 50-55%, 55-60%, 60-65%, and 65-70% purity (e.g., non-progenitor cells have been removed or are otherwise absent from the population). More preferably the purity is about 70-75%, 75-80%, 80-85%; and most preferably the purity is about 85-90%, 90-95%, and 95-100%.

Counter filtration is a technique that can be used to isolate nerve fibers from adipose tissue, for example, SAT or VAT after enzymatic or mechanical digestion. Enzymes such as dispase and collagenase can be used for the process of enzymatic digestion. The collagenase can be selected from collagenase type I, collagenase type IA, collagenase type II, collagenase type III, collagenase type IV, collagenase type XI, collagenase type A, collagenase type B, collagenase type C, collagenase type F, collagenase A, collagenase B, collagenase D, collagenase H, collagenase I, collagenase II, collagenase P, collagenase/dispase, liberase, matrix metallopeptidase-1/fibroblast collagenase/interstitial collagenase (MMP-1), MMP-2, MMP-8/neutrophil collagenase, MMP-9, MMP-13/collagenase 3, or MMP-18/collagenase 4. In some embodiments, the collagenase is collagenase type XI. In some embodiments, the collagenase is liberase which contains collagenases. Counter-filtered material is obtained after dissociated SAT or VAT is filtered through a cell strainer that can range in size from 20 μm to 140 μm, such as 20 μm, 30 μm, 40 μm, 50 μm, 60 μm, 70 μm, 80 μm, 90 μm, 100 μm, 110 μm, 120 μm, 130 μm, or 140 μm, and it is a technique that can be used to enrich NFBs. In some embodiments, the cell strainer is a 70 μm cell strainer. NFBs can be identified visually from counter-filtered material. Following counter-filtration, NFBs can be identified using several dyes such as fluoromyelin red, styryl pyridinium dyes FM1-43 and AM1-43, thiazin dyes etc. Fluoromyelin red is a fluorescent myelin stain and helps stain the myelin sheath which serves as insulation for nerve fibers (see e.g., Huval et al., Lab Chip, 2015, 15, 2221-2232). NFBs are known to stain with the non-toxic dye fluoromyelin red which serves as a method of validating the success of the counter filtration technique in enriching NFBs.

Purity of the neuronal progenitors can be determined according to the desired genetic marker profile within a population. Neuronal progenitor cells of the invention express at least Plp1, P75, and SOX10 or P75 and CD49f. Plp1, P75, and SOX10 and P75 and CD49f expressing neural progenitor cells of the invention are produced from SAT or VAT. Neuronal progenitor cells of the invention do not express Zic1 or do not express a significant amount of Zic1. For an amount of gene expression to be significant, at least 1 count per 1 million counts (CPM) of the mRNA transcript must be detected in a sample. Expression of Sox9, Msx1, Msx2, Nes, and Pax3, which can be prominent among established neuronal stem cell lines (Li et al., 2018), was not significant among neuronal progenitor cells of the invention. Expression of Sox9, Msx1, Msx2, and Pax3 was essentially indistinguishable between neuronal progenitor cells of the invention and heterogenous cells of the adipose tissue, e.g., SAT or VAT (i.e., non-neural progenitors).

Plp1 is a glial/neuronal progenitor biomarker known as proteolipid protein 1, which is the primary constituent of myelin in the central nervous system. The human gene encoding Plp encodes a 276-amino acid polypeptide with 5 strongly hydrophobic domains that interact with the lipid bilayer as trans- and cis-membrane segments. Diehl, Schaich, Budzinski, and Stoffel (1986) determined that the human Plp gene contains 7 exons and spans approximately 17 kb. Quantification of Plp1 expressing cells can be done via flow cytometry and immunohistochemistry. Expression of the human Plp1 can be assayed by PCR using, for example, the RNeasy Micro Kit (Qiagen). The measurement was made as described in Example 11 below. PCR can be used to validate the samples as it detects expression at the gene (mRNA) level in cell lysates. The human PLP1 protein can also be assayed using enzyme-linked immunosorbent assay (ELISA), for example, the “human proteolipid protein 1, myelin (PLP1) ELISA kit”. Such assays generally have high sensitivity and excellent specificity for protein detection. Typically, ELISAs are used to quantify secreted proteins. They can be used to measure proteins like PLP1 in a cell lysate.

Neuronal biomarker nerve growth factor receptor (NGFR) is also referred to as p75 neurotrophin receptor (P75 (NTR)) because of its molecular mass and its ability to bind at low affinity to not only NGF, but also other neurotrophins such as neurotrophin-3, neurotrophin-4, and brain-derived neurotrophic factor. It is a low affinity nerve growth factor receptor and is important for developing neurons, refinement of neuronal connections, neuronal survival, and death. Human sequence information for P75 is known in the art (Johnson et al., 1986; Ota et al., 2004). Quantification of P75 expressing cells can be done via immunohistochemistry, via PCR, for example, using the RNeasy Micro Kit (Qiagen), to measure expression at the gene (mRNA) level in cell lysates as described in Example 11 below, or via using ELISA kits such as the Biosensis NGFR/p75ECD ELISA kit on cell lysates.

Neuronal biomarker SRY-box transcription factor 10 (SOX10) is a transcription factor that functions in neural crest, peripheral nervous system (PNS) and oligodendrocyte development by acting as a nucleocytoplasmic shuttle protein. It plays a major role in embryonic development and cell fate determination. This protein is also important for formation of nerves in the enteric nervous system (ENS) such as the nerves in the intestine. Human sequence information for SOX10 is known in the art (Pusch et al., 1998). Quantification of SOX10 expressing cells can be done via immunohistochemistry, via PCR, for example, using the RNeasy Micro Kit (Qiagen), to measure expression at the gene (mRNA) level in cell lysates as described in Example 11 below, or by using a human SOX10 ELISA kit on cell lysates. CD49f is an alias for the protein integrin alpha-6 (ITGA6). In humans, it is encoded by the ITGA6 gene. It is a 120 kD transmembrane protein and an integral cell surface protein composed of an alpha chain and a beta chain. CD49f has been identified in more than 30 stem cell populations and plays a role in maintaining and regulating self-renewal in stem cells. Quantification of CD49f expressing cells can be done via immunohistochemistry. It can also be quantified via PCR, for example, using the RNeasy Micro Kit (Qiagen), to measure expression at the gene (mRNA) level in cell lysates as described in Example 11 below or by using a Human ITGA6/Integrin Alpha 6/CD49f (Sandwich ELISA) ELISA kit on cell lysates. Zic Family Member 1 or Zinc Finger Protein ZIC 1 (Zic1) acts as a transcriptional activator and is involved in neurogenesis. Zic1 plays important roles in the early stages of organogenesis of the central nervous system (CNS), as well as during dorsal spinal cord development and maturation of the cerebellum. Human sequence information for Zic 1 is known in the art (NCBI RefSeq Accession: NM_003412.4) (O'Leary et al., 2016). Zic1 can be quantified via PCR, for example, using the RNeasy Micro Kit (Qiagen), to measure expression at the gene (mRNA) level in cell lysates as described in Example 11 below, by using a Human Zinc Finger Protein ZIC 1 (ZIC1) ELISA kit on cell lysates, or by immunohistochemistry using Zic1 antibodies.

Sox9 is a transcription factor that plays a key role in chondrocytes differentiation and skeletal development. Human sequence information for Sox 9 is known in the art (NCBI RefSeq Accession: NM_000346.4) (O'Leary et al., 2016).

Msx1 acts as a transcriptional repressor that plays a role in craniofacial development and specifically, in odontogenesis. Human sequence information for Msx1 is known in the art (NCBI RefSeq Accession: NM_002448.3) (O'Leary et al., 2016).

Msx2 acts as a transcriptional regulator in bone development. Human sequence information for Msx2 is known in the art (NCBI RefSeq Accession: NM_002449.5 and NM_001363626.2) (O'Leary et al., 2016). Pax3 is a transcription factor that can regulate cell proliferation, migration, and apoptosis. It is involved in neural development and myogenesis. Human sequence information for Pax3 is known in the art (NCBI RefSeq Accession: NM_181460.4, NM_181459.4, NM_181458.4, NM_001127366.3, NM_181461.4 and NM_181457.4) (O'Leary et al., 2016).

The present invention is additionally described by way of the following illustrative, non-limiting examples that provide a better understanding of the present invention and of its many advantages.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

The materials and methods used to conduct the assays in the following examples are described in detail herein below.

Subcutaneous adipose tissue-neural stem cell (SAT-NSC) isolation from mice: Posterior SAT fat pads were dissected from 1-month old mice euthanised via CO₂ inhalation. SAT was immediately minced and incubated with Collagenase XI (1 mg/mL; Sigma Aldrich, St. Louis, MO) and Dispase (250 μg/ml; StemCell Technologies, Vancouver, BC) solution for 40 min at 37° C. and triturated every 10 min for dissociation. Tissue suspensions were centrifuged at 500G for 5 minutes and the supernatant was discarded to remove the excess lipid. The solution was resuspended in neurobasal medium and either filtered through a 40 μm cell strainer to generate single cell suspensions or left unfiltered to retain the nerve fibre bundles (NFBs).

Cell solutions were seeded into 6 well low attachment culture dishes at a density of 5×10⁵ cells in 2 ml of neuroproliferation medium consisting of Neurocult Mouse Proliferation Supplement (10%, StemCell Technologies), basic fibroblast growth factor (20 ng/ml, StemCell Technologies), epidermal growth factor (20 ng/ml, StemCell Technologies), heparin (0.0002%, StemCell Technologies) and penicillin and streptomycin (1%, Gibco, Life Technologies) in Neurocult Mouse Basal Medium (StemCell Technologies). Cells were cultured in humidified incubator with 5% CO², atmospheric oxygen at 37° C. Low media volumes were used to assist with initial cell aggregation. An additional 2 mL of media added after 3 days and cells were cultured for 10 days at which point numerous spheroids were observed.

Spheroid containing solutions were transferred to fibronectin-coated (1:500 of sterile PBS for 2 h at 37° C.) cell culture flasks seeded (7-7.5 cm² per mL of cell solution) and supplemented with 5% FBS to promote attachment and cell migration from spheroids. After 48 h, cells were washed with PBS and trypsinised with TrypIE Select Enzyme (Gibco, Life Technologies) for 5 minutes at 37° C. Trypsinization was neutralised with 1:2 volume of basal media containing 2.5% FBS and cells were pelleted via centrifugation as described above. Cells were resuspended in neuroproliferation medium and stained with DAPI (1:1000) for fluorescence activated cell sorting (FACS) of either Wnt1-tdT or Plp1-GFP expressing cells from transgenic reporter mice. Purified cells obtained via FACS were plated at a density of 1000 cells/cm² in 24-well low attachment plates in culture conditions and media as described above to generate homogenous neurospheres.

NFB isolation and culture from human abdominal SAT: Human abdominal SAT was stored overnight at 4° C., cut into ˜1 cm² pieces and washed 3 times in sterile PBS. For enzymatic digestion, pieces of SAT were digested in Liberase™ Thermolysin High formulation (25 μg/mL, Roche) and Dispase (0.05 U/ml; StemCell Technologies, Vancouver, BC) for 3 h in a humidified incubator at 37° C. Digested SAT was aliquoted into C-tubes (Miltenyi Biotec) for homogenisation using the GentleMACs (program: spleen 1, Miltenyi Biotec) tissue dissociator system (enzymatic and mechanical digestion). In addition, a second method utilizing only mechanical digestion as detailed above without prior incubation in the enzymatic cocktail was performed. The dissociated SAT was filtered through a 70 μm cell strainer, and the counter-filtered material was collected and washed with sterile PBS. Under a light dissection microscope, NFBs were manually collected based on morphology, with NFBs exhibiting characteristic striations and ‘frayed’ edges observed at the sites where the NFBs had been severed.

NFBs were cut into 1-2 mm length pieces and placed into low-attachment culture dishes (24-well) containing 1 mL of human neural proliferation medium (basic fibroblast growth factor (20 ng/mL, StemCell Technologies) epidermal growth factor (20 ng/mL, StemCell Technologies) heparin (0.0002%, StemCell Technologies), GlutaMAX (1%, Gibco, Life Technologies), B27 supplement (1%), Primocin, (1%), Metronidazole (50 μg/mL) and FBS (5%) in Dulbecco's Modified Eagle Medium: Nutrient Mixture F-12 (DMEM/F12, Gibco, Life Technologies). After 3 days the media was doubled and by day 7 NFBs isolated by enzymatic and mechanical digestion had remodeled into spheroids.

Following 10 days of culture, spheroids were transferred to tissue culture treated 6 well plates coated with fibronectin with additional media added at a ratio of 2:3. After 2 weeks, cells from the NFB derived spheroids had migrated onto the surface of the cell culture flask. Cells were trypsinized as described above yielding between 1.5-8.5×10⁴ cells per NFB-derived spheroid and were passaged and expanded by replating in 24-well low-attachment tissue culture plates at density of 2.5×10³ cells/cm² in human neuroproliferation medium with weekly replacement of half of the media over 4 weeks.

### Example 1. Neural Crest (NC)-Derived Schwann Cells Reside in NFBs Coursing Through the SAT

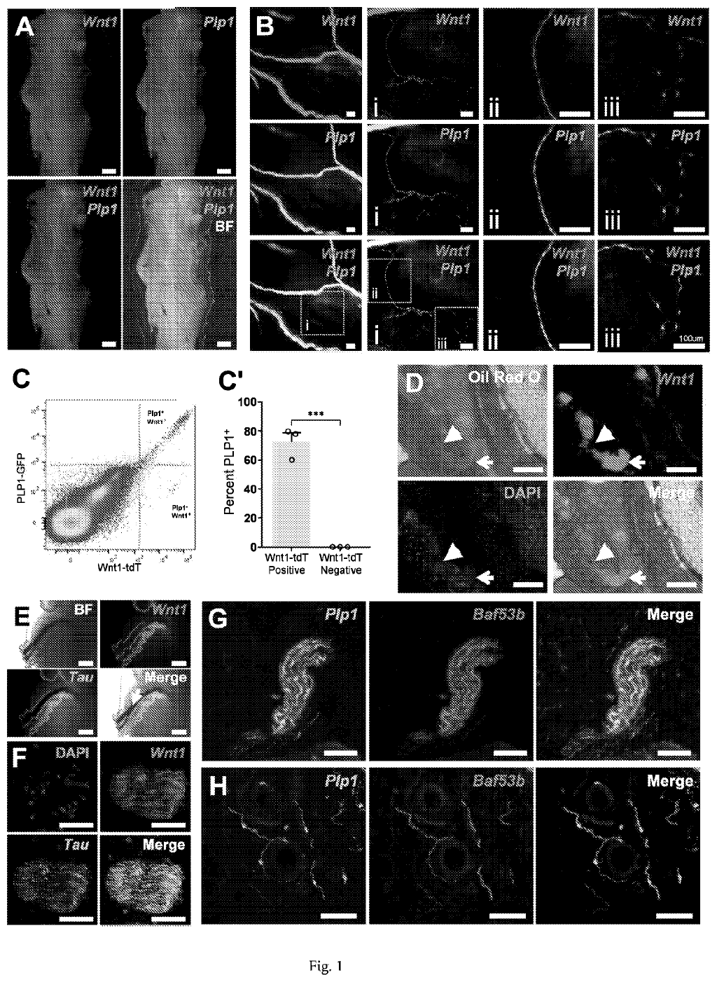

The NC gives rise to all peripheral neurons and glial cells, including Schwann cells. The constituents of neural-crest derived structures (Wnt1⁺) and Schwann cells (Plp1⁺) in the posterior subcutaneous fat pads were examined in Wnt1::tdT; Plp1-GFP mice. Large Wnt1⁺ Plp1⁺ fibre-like structures were observed throughout the SAT (FIG. 1 Panel A). These structures branched several times until individual Wnt1⁺ Plp1⁺ cells were observed (FIG. 1 Panel B). Wnt1⁺ Plp1⁺ cells exhibited a predominantly bipolar morphology and formed physical connections end-to-end, consistent with a Schwann cell identity. In single cell suspensions of the SAT, 72.6±5.8% of Wnt1⁺ cells expressed the glial cell marker Plp1, which was not present in the Wnt1-cell population (FIG. 1 Panel C-Panel C′). In cross sections of the SAT, large Wnt1⁺ fibres containing cells were observed to penetrate deep within the adipose (FIG. 1 Panel D) and could be observed entering the adipose alone, or alongside blood vessels (FIG. 1 Panel E). Wnt1 expression overlapped with the neuronal marker Tau-GFP, confirming that these structures were NFBs (FIG. 1 Panel E). Within NFBs, Wnt1⁺ cells and Wnt1⁺ Tau⁺ nerve fibre processes were observed (FIG. 1 Panel F). Importantly, only Wnt1⁺ cells and no Wnt1⁺ Tau⁺ cells were observed in single cell suspensions of digested SAT, indicating that Wnt1⁺ Tau⁺ nerve fibres originate extrinsically to the SAT. Using the Baf53b::tdT neuronal reporter mouse, Plp1⁺ cells were confirmed to be closely juxtaposed to Baf53b⁺ nerve fibres, which did not originate from cell bodies within the SAT, and is consistent with a supporting role of Plp1⁺ cells for extrinsic nerve fibres in NFBs (FIG. 1 Panel G) and individual nerve fibre processes throughout the SAT (FIG. 1 Panel H).

### Example 2. NSCs in the SAT Originate from NC-Derived Schwann Cells

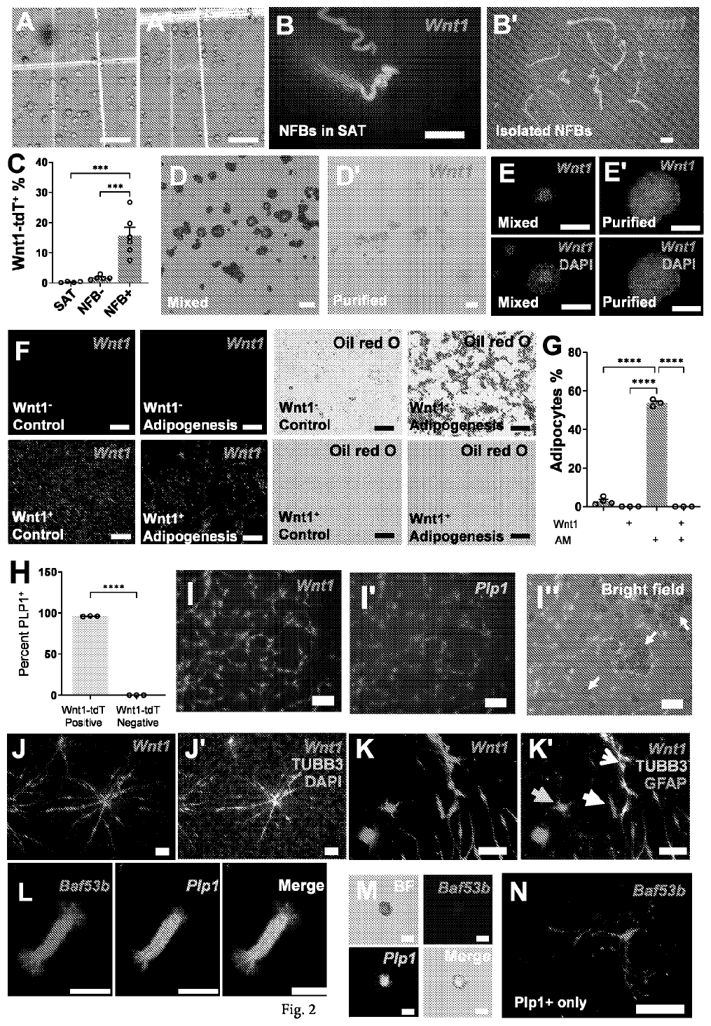

NC-derived (Wnt1⁺) cells from the SAT were isolated from enzymatically digested SAT. While occasional Wnt1⁺ cells assumed a spherical morphology after digestion (FIG. 2 Panel A), the majority retained their characteristic bipolar structure, unlike Wnt1⁻ cells (FIG. 2 Panel A′). NFBs from the SAT were resistant to enzymatic digestion and are removed after traditional filtration procedures to produce single cell suspensions of the SAT (FIG. 2 Panel B-Panel B′). Only 0.27±0.09% of nucleated cells from the SAT were Wnt1⁺ (FIG. 2 Panel C). Filtered cells, which excludes the NFBs, from the SAT cultured in low attachment conditions with neuroproliferation medium formed spheroids in vitro and increased the proportion of Wnt1⁺ cells ˜6.5 fold (FIG. 2 Panel C). Cells seeded without prior filtration and cultured in the same conditions also formed spheroids with a ˜57.8 fold enrichment of Wnt1⁺ cells, confirming that the majority of NC-derived cells originate from NFBs (FIG. 2 Panel C). Individual spheroids were heterogenous with Wnt1⁺ positive cells primarily occupying the centre of spheroids (FIG. 2 Panel D, FIG. 2 Panel E). Purified Wnt1⁺ cells isolated from heterogenous spheroids were capable of reforming spheroids and proliferating without the support of Wnt1⁻ cells (FIG. 2 Panel D′, FIG. 2 Panel E′).

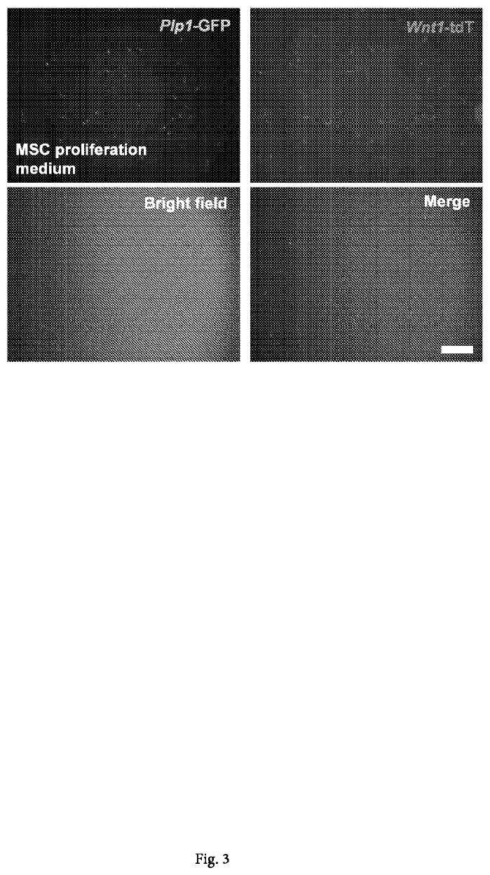

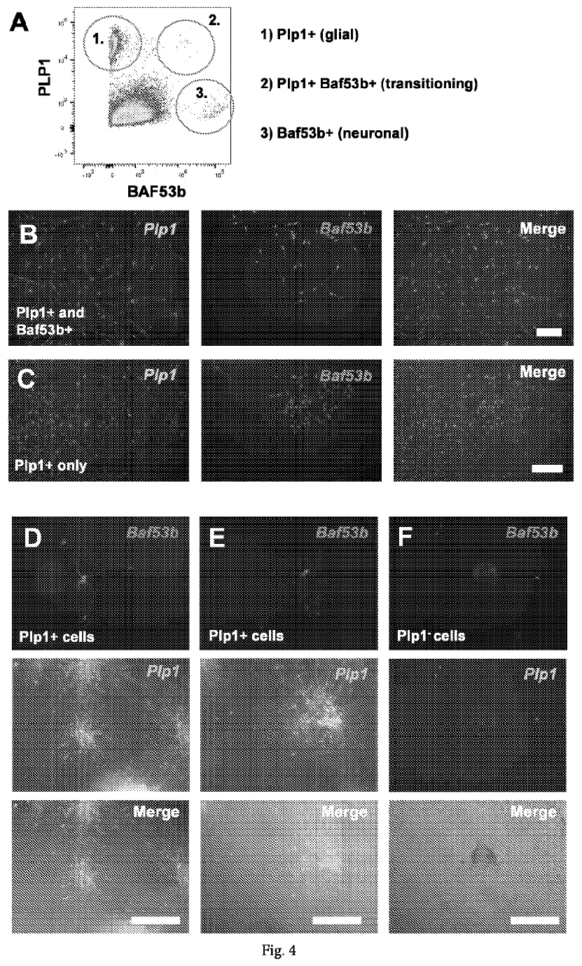

Previously, it was suggested that the NC gives rise to preadipocytes in the SAT (Sowa et al., 2013). An adipogenesis assay was conducted using purified populations of Wnt1⁺ and Wnt1⁻ cells isolated from heterogenous spheroids (FIG. 2 Panel F). The proportion of Oil Red O⁺ adipocytes was significantly elevated in Wnt1-cultures exposed to control and adipogenesis induction medium (AM) compared to those containing Wnt1⁺ cells (FIG. 2 Panel G). In fact, adipogenesis was not detected in Wnt1⁺ cultures in either culture condition (FIG. 2 Panel G). In dissociated spheroids, 96.0±0.2% of Wnt1⁺ cells expressed the glial cell marker Plp1 which was not detected in the Wnt1-population, indicating enrichment of NC-derived glial cells (FIG. 2 Panel H). When the adipogenesis assay was performed using heterogenous cells, no Wnt1⁺ adipocytes were observed and Plp1 was expressed by nearly all Wnt1⁺ cells, indicating that NC-derived cells from the SAT favor a glial phenotype (FIG. 2 Panel I). Furthermore, when SAT was digested and cultured using methods intended for mesenchymal stem cell (MSC) isolation, dual Wnt1⁺ Plp1⁺ cells were observed to persist in MSC culture media and contaminated cultures (FIG. 3). Purified Wnt1⁺ cells were responsive to neural differentiation medium, forming TUBB3⁺ neurons and GFAP⁺ glial cells, suggesting that Wnt1⁺ cells contain a NSC population and that these spheroids may be considered neurospheres (FIG. 2 Panel J). Using Plp1; Baf53b glial-neuron reporter mice, expression of Plp1 and Baf53b was observed in SAT NFBs (FIG. 2 Panel K); however Baf53b expression was dramatically reduced during spheroid culture (FIG. 2 Panel L). Production of tdT cannot be muted after cre-lox recombination in this model; therefore, the NFBs were devoid of Baf53b⁺ neuronal bodies and stopped expressing tdT after the neuronal fibres from extrinsic sources were severed. Analysis by flow cytometry revealed a large number of Plp1⁺ cells with smaller populations of Baf53b⁺ and dual Plp1⁺Baf53b⁺ cells in heterogenous neurospheres (FIG. 4). The presence of Baf53b⁺ and dual Plp1⁺Baf53b⁺ cell populations was validated by in vitro culture on fibronectin. Furthermore, both of these cell populations arose from purified Plp1⁺ cells in monolayer cultures (FIG. 4). When Plp1⁺ and Plp1 cells were purified and cultured separately in neuronal differentiation conditions, Plp1⁺ cells could give rise to morphologically distinct Baf53b⁺ neurons with multiple neural processes, but these were not observed in Plp1-negative cultures (FIG. 2 Panel M, FIG. 4). Thus, NC-derived Schwann cells are able to give rise to SAT-NSCs.

### Example 3. In Vitro Culture of Schwann Cells Induces Reprogramming Pathways to Generate SAT-NSCs

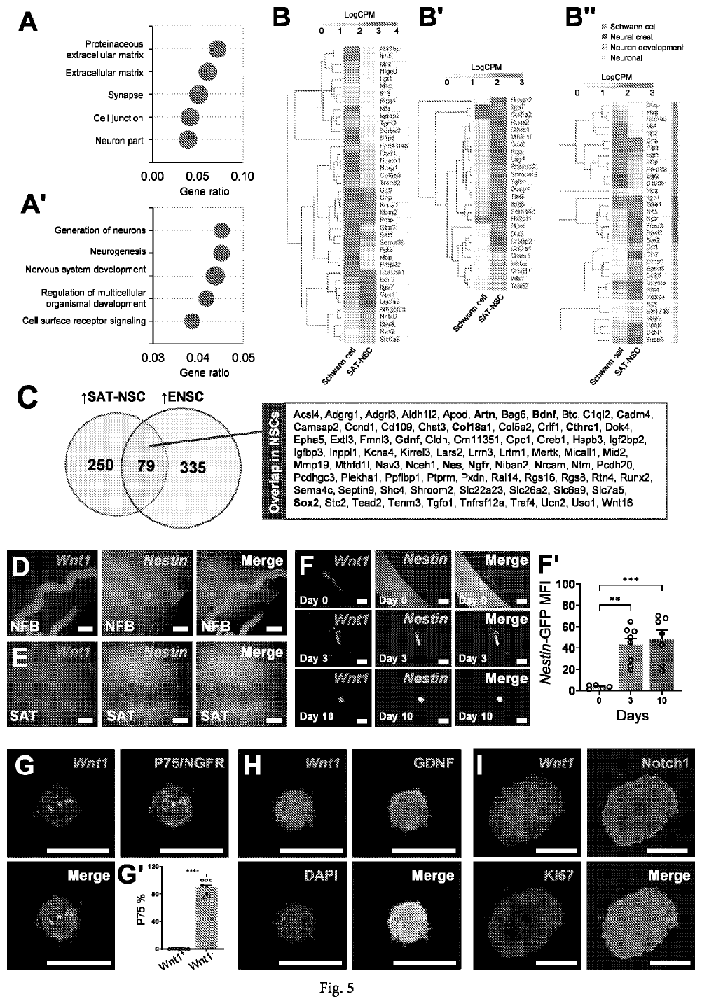

As no neuronal cell bodies are present in the SAT, these Schwann cells appear to acquire their neurogenic potential via reprogramming in vitro. To examine the transition of SAT Schwann cells to SAT-NSCs, their transcriptomes were compared. A total of 329 genes were upregulated in SAT-NSCs compared to Schwann cells, with an enrichment of genes associated with neurogenesis and nervous system development processes (FIG. 5 Panel A, FIG. 5 Panel A′). The NC-derived SAT cells were validated as Schwann cells by demonstrating their high level of expression of Schwann cell markers, as shown in FIG. 5 Panel B, generated from a published SAT transcriptomic dataset (Rajbhandari et al., 2019). These genes were largely downregulated after generation of SAT-NSCs, including genes encoding myelin proteins such as Mpz (−451 fold), Pmp22 (−281 fold), Mal (−222 fold) and Mbp (−197 fold). The loss of Schwann cell properties indicated that SAT-NSCs may be formed via mechanisms of dedifferentiation. This was reinforced by analysis indicating that differentially expressed genes (DEGs) upregulated in SAT-NSCs are more enriched for embryonic properties (26/329, 7.90%) when compared to DEGs upregulated in Schwann cells (39/1121, 3.47%; Chi-squared test, p<0.001) (FIG. 5 Panel B′). Further examination identified downregulation of Schwann cell markers (Cnp, −8 fold; Egr2, −13 fold and S100b, −5 fold) (FIG. 5 Panel B″); and an upregulation in neurogenic markers, including Uchl1 (PGP9.5, 56 fold), Tubb3 (TUBB3, 5 fold) and Map2 (3 fold), suggesting that SAT-NSCs are capable of deviating from a Schwann cell restricted fate (FIG. 5 Panel B″). Furthermore, the expression of several neural crest stem cell markers were upregulated, including Nes (57 fold), Ngfr (P75, 36 fold) and Sox2 (9 fold) (FIG. 5 Panel B″). Notably, these features were consistent with NSCs isolated from the enteric nervous system, with 24% of genes upregulated in SAT-NSCs shared between these cell types (FIG. 5 Panel C). To validate the ability of Schwann cells to acquire stem cell properties, the expression of the NSC marker Nes was studied in Nestin^{GFP}; Wnt1: Cre; ROSA26^{tdTomato} (Nestin^{GFP}; Wnt1-tdT) reporter mice. In the SAT, Wnt1-tdT and Nestin^{GFP} were restricted to separate cell populations in NFBs (FIG. 5 Panel D) and the surrounding tissue (FIG. 5 Panel E). Likewise, NFBs isolated from the SAT exhibited minimal expression of Nestin (FIG. 5 Panel F). After 3 days in culture, expression of Nestin was induced in the NFBs, which formed spheroids by 10 days and continued to exhibit high Nestin expression, consistent with gene expression data (FIG. 5 Panel B″-Panel C, FIG. 5 Panel F′). Similarly, the expression of the NC-derived neural stem cell marker P75 (NGFR) was confirmed on the protein level in heterogenous spheroids (FIG. Panel G). P75 colocalized with the majority of Wnt1⁺ cells with minimal expression in the Wnt1⁻ cells, indicating its high specificity as a SAT-NSC marker (FIG. 5 Panel G′). In purified neurospheres, expression of GDNF was validated by immunohistochemistry (FIG. 5 Panel H). SAT-NSCs expressed the neuronal-glial fate regulation receptor Notch1 and the cell proliferation marker Ki67 in the periphery of the spheres (FIG. 5 Panel I).

### Example 4. SAT-NSCs Engraft in the Colorectal Wall Following Cell Transplantation In Vivo and Differentiate into Neurons

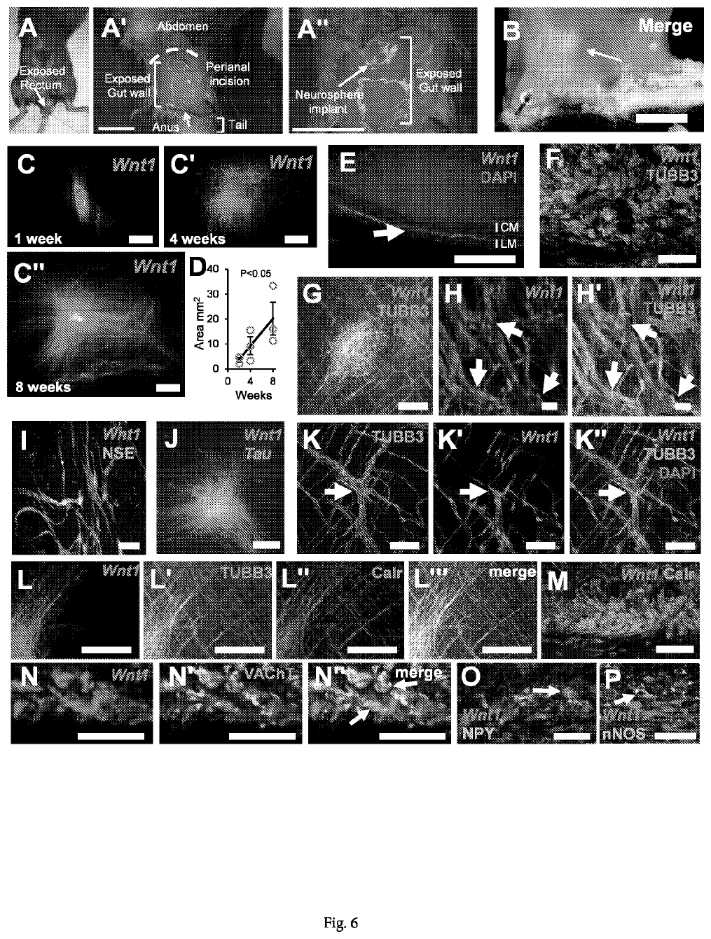

Considering that NSCs from the NC give rise to enteric neurons in the embryonic environment and Schwann cells can give rise to enteric neurons postnatally (El-Nachef & Bronner, 2020; Uesaka, Nagashimada, & Enomoto, 2015), the fate of SAT-NSCs in the gut environment in vivo was examined. Neurospheres generated from Wnt1⁺ cells were implanted microsurgically into the smooth muscle wall of the colorectum of adult mice (FIG. 6 Panel A-Panel A″). All neurospheres generated from purified Wnt1⁺ cells survived, engrafted, and migrated within the intestinal wall of allogeneic recipients (n=9/9 mice; FIG. 6 Panel B). The area covered by implanted SAT-NSCs increased gradually with time post-transplantation, with a 20.17±6.74 mm² coverage area after 8 weeks from single implanted neurospheres (FIG. 6 Panel C-Panel D). In cross sections of the colon, SAT-NSCs were confirmed to engraft into the appropriate layer of the myenteric plexus, between the circular and longitudinal muscle (FIG. 6 Panel E), with many transplanted cells expressing the neuronal marker TUBB3 after 8 weeks (FIG. 6 Panel F). Recipients had high rates of Wnt1⁺ cell survival and neural differentiation, indicated by immunoreactivity for the neuronal marker TUBB3 in wholemount preparations of the smooth muscle (FIG. 6 Panel G). Transplanted cells formed ganglia-like structures containing neurons (FIG. 6 Panel H-Panel H′) as evidenced by their immunoreactivity for TUBB3, neuron specific enolase (NSE; FIG. 6 Panel I) and the induction of transgenic Tau^{GFP} expression in SAT-NSCs isolated from Tau^{GFP}; Wnt1-tdT mice (FIG. 6 Panel J).

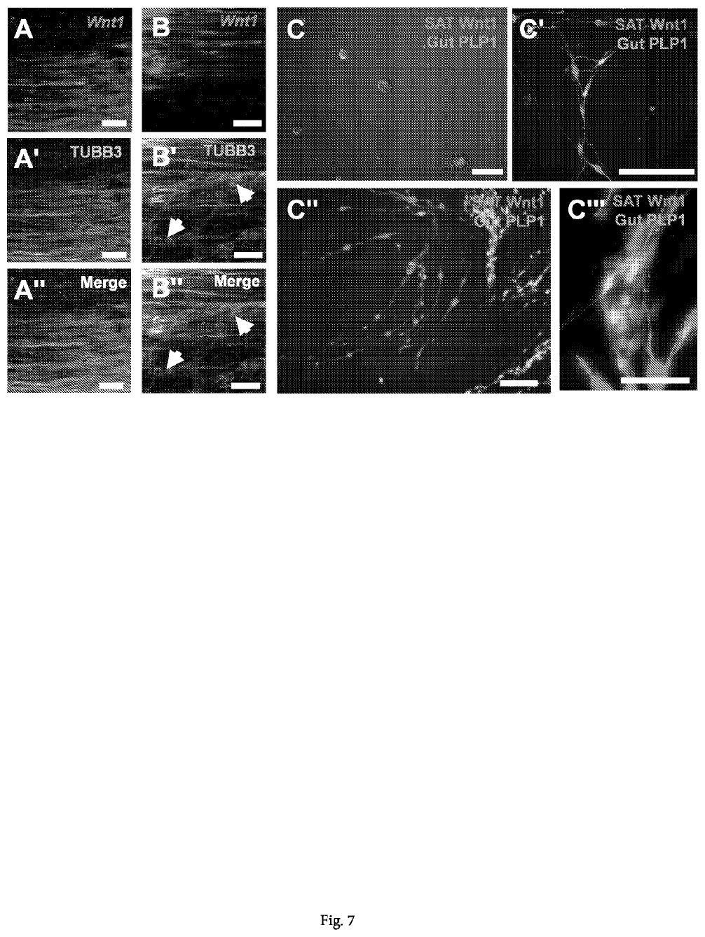

Transplanted Wnt1⁺ cells formed physical connections with the host ENS and incorporated into myenteric ganglia containing both endogenous TUBB3 immunoreactive cells and transplanted Wnt1⁺ cells (FIG. 6 Panel K-Panel K″). Neuronal subtype differentiation consistent with an enteric neuronal phenotype was observed at 8 weeks post transplantation with Wnt1⁺ cells expressing calretinin (Calr), the vesicular acetylcholine transporter (VAChT) and neuropeptide Y (NPY) (FIG. 6 Panel L-Panel O). We did not detect immunoreactivity to Substance P, CGRP, ENK and TH, suggesting that SAT-NSCs may not recapitulate all enteric neuronal subtypes at the timeframes examined. In similar experiments, SAT-NSC neurospheres from Wnt1-tdT mice survived and thrived after implantation into muscularis propria preparations of the distal colon from wild-type recipient mice ex vivo. Extensive migration of Wnt1⁺ was observed within the smooth muscle wall (FIG. 7 Panel A). After 7 days, Wnt1⁺ transplanted cells were observed to express the enteric neuron marker TUBB3 (FIG. 7 Panel A-Panel A″) and to integrate with endogenous TUBB3 immunoreactive ganglia of the myenteric plexus (FIG. 7 Panel B-Panel B″). This was validated in co-culture experiments with mixed cell spheroids from the SAT of Wnt1-tdT mice and the colonic muscularis propria of Plp1^{GFP} mice (FIG. 7 Panel C). Wnt1⁺ SAT-NSCs and Plp1⁺ enteric glial cells aggregated into ganglia-like complexes in vitro and long Wnt1⁺ fibres were observed to project through enteric Plp1⁺ ganglia, suggesting an affinity between SAT-NSCs and ENS cells (FIG. 7 Panel C-Panel C″). Evidence of enteric neuronal subtype differentiation was observed in these organ cultures with expression of NOS1 by transplanted SAT-NSCs (FIG. 6 Panel P).

### Example 5. SAT-NSC Transplantation Ameliorates Gastroparesis

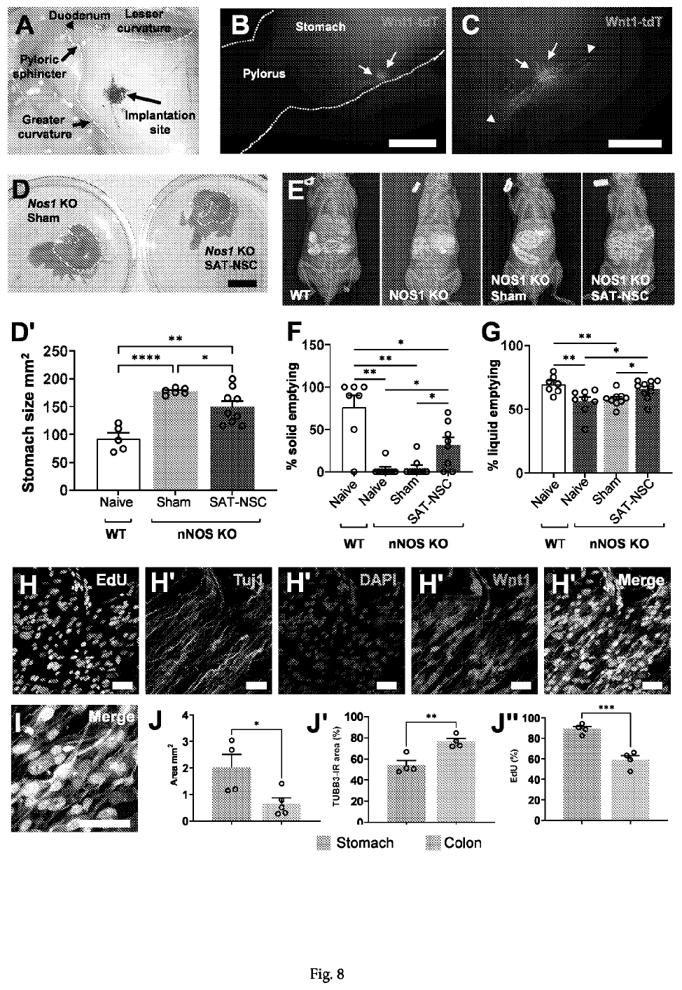

The therapeutic potential of SAT-NSCs to treat neurogastrointestinal disorders was evaluated in mouse models of gastroparesis and Hirschsprung disease (HSCR). To examine the effects of SAT-NSC transplantation on gastric emptying in a model of gastroparesis, neurospheres were implanted into the stomach wall of nNOS^{-/-} mice (FIG. 8 Panel A). Cells were administered via laparotomy and microsurgical implantation of neurospheres into the muscularis externa of the gastric antrum, just proximal to the pylorus (FIG. 8 Panel A). SAT-NSCs engrafted in the stomach (FIG. 8 Panel B) and projected nerve fibres (FIG. 8C) 10 weeks post-implantation. SAT-NSC implantation partially restored normal gastric size as compared to the significant enlargement of the stomach normally observed in nNOS^{-/-} mice (FIG. 8 Panel D-Panel D′). Radiographic gastric emptying assays were performed to assess the effects of SAT-NSCs on the emptying of solid and liquid material that was gavaged to the stomach (FIG. 8 Panel E). Interestingly, SAT-NSC implantation significantly improved gastric emptying of solids compared to naïve and sham-treated nNOS KO mice, indicating an amelioration of gastroparesis (FIG. 8 Panel F). Similarly, SAT-NSCs restored liquid emptying in nNOS KO mice to near normal levels (FIG. 8 Panel G). Considering the intestinal milieu appears to promote neural differentiation of SAT-NSCs, the effects of the differing microenvironments of the colon and stomach on the properties of SAT-NSCs were assessed.

Neurospheres were transplanted to ex vivo tissue preparations of colon and gastric antrum from the same recipients and were cultured for 7 days to assess migration, proliferation by EdU incorporation, and neural differentiation by immunohistochemistry for TUBB3 (FIG. 8 Panel H-Panel I). Interestingly, the microenvironment of the antrum was preferential toward SAT-NSC spreading (FIG. 8 Panel J), with less neuronal differentiation (FIG. 8 Panel J′) and higher cell proliferation than SAT-NSCs transplanted into the colon (FIG. 8 Panel J″), which indicates that the properties of SAT-NSCs are dependent on the recipient tissue microenvironment.

### Example 6. SAT-NSC Transplantation Restores Muscle Neurally-Mediated Muscle Contraction in the Colon

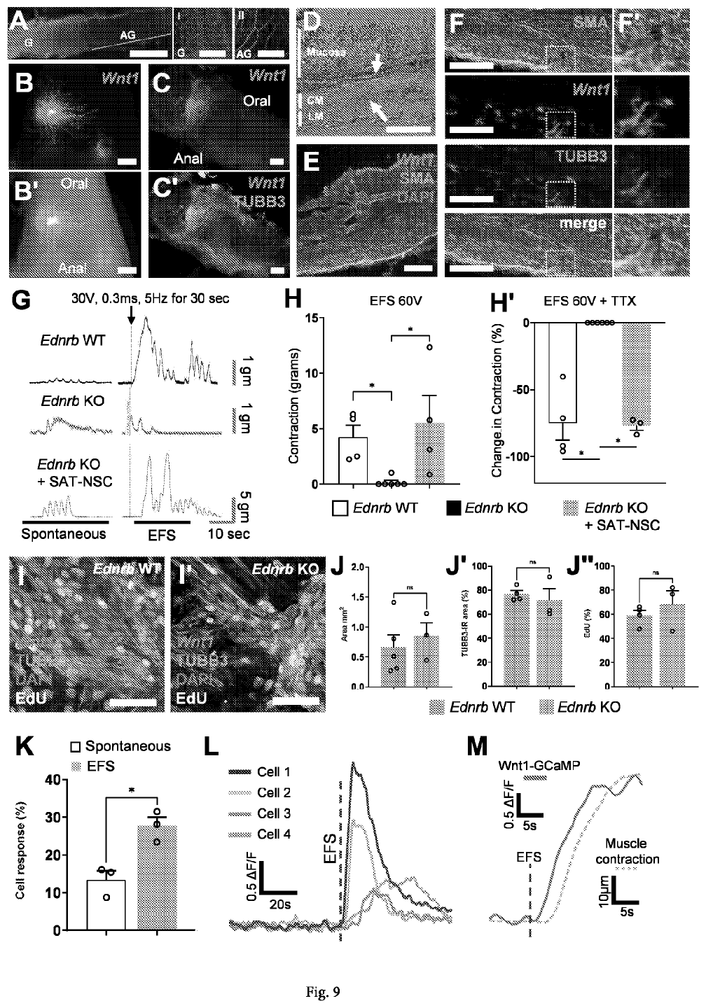

To examine the effects of SAT-NSC in HSCR, neurospheres were implanted to the colorectum of Ednrb^{-/-} mice with distal colonic aganglionosis. As demonstrated in the Wnt1-tdT; Ednrb/transgenic mouse (FIG. 9 Panel A), the Ednrb null mutation results in intestinal aganglionosis in the mid colon to the rectum with hypertrophic nerve fibres from extrinsic sources present in the aganglionic segment, typical of short-segment human HSCR. SAT-NSC neurospheres were observed to engraft and spread throughout the aganglionic environment without the support of a pre-existing ENS 2-3 weeks after administration (FIG. 9 Panel B). Immunohistochemical labelling of wholemount tissue preparations indicated that SAT-NSCs differentiated into neurons and resided near hypertrophic nerve fibres in the aganglionic segment (FIG. 9 Panel C). SAT-NSCs were observed to migrate between the mucosa and circular muscle, and between the circular and longitudinal muscle layers, in the aganglionic region. These represent the normal locations of the submucosal and myenteric plexuses, respectively, in normal ganglionated colon (FIG. 9 Panel D-Panel E). Likewise, several SAT-NSCs differentiated into neurons in the muscularis (FIG. 9 Panel F). Mice were assessed for fecal pellet production over 1 hour prior to sacrificing. Pellet output was observed in 3/5 Ednrb^{-/-} mice with SAT-NSC transplants compared to 0/5 Ednrb^{-/-} non-transplanted controls. In smooth muscle contraction experiments, colonic tissue collected from Ednrb^{-/-} mice exhibited no response to electric field stimulation (EFS) in stark contrast to wildtype littermates (FIG. 9 Panel G-Panel H). These contractile properties were restored in the colonic regions of Ednrb-mice transplanted with SAT-NSCs (FIG. 9 Panel G-Panel H). Application of tetrodotoxin (TTX) negated EFS-evoked contractile responses in the Ednrb WT and Ednrb/mice with SAT-NSC transplants, indicating that these responses were neurally-mediated (FIG. 6 Panel H′). In ex vivo preparations of ganglionic and aganglionic colon, SAT-NSCs engrafted and exhibited equivalent extent of migration, TUBB3 immunoreactivity, and cell proliferation in both microenvironments (FIG. 9 Panel I-Panel J″). Calcium imaging studies using SAT-NSCs derived from Wnt1::Cre; Polr2a^{GCaMP5g-tdTomato} (Wnt1-GCaMP5-tdT) mice demonstrated that SAT-NSCs transplanted to the aganglionic environment directly respond to EFS by elevating [Ca²⁺], consistent with functional neurons (FIG. 9 Panel K, Movie S1). Transplanted cells exhibited a fast upstroke in calcium influx immediately upon EFS, followed by a biexponential decay consistent with the properties of enteric neurons (FIG. 9 Panel L, cells 1-2) (Boesmans et al., 2013).

Likewise, many cells exhibited a delayed gaussian-like response to EFS similar to secondary responses observed in enteric glia (FIG. 9 Panel L, cells 3-4) (Boesmans et al., 2013). Global calcium responses to EFS occurred in transplanted cells immediately preceding the onset of muscle contraction (FIG. 9 Panel M). Given the absence of endogenous neurons in Ednrb^{-/-} distal colon, this suggests that SAT-NSCs are able to mediate muscle contraction in the aganglionic segment. Together, the data in Ednrb^{-/-} and nNOS^{-/-} mice demonstrate the potential benefit of SAT-NSCs for the treatment of neurointestinal disease in both aganglionic and ganglionated environments.

### Example 7. Human SAT NFBs Contain NSCs

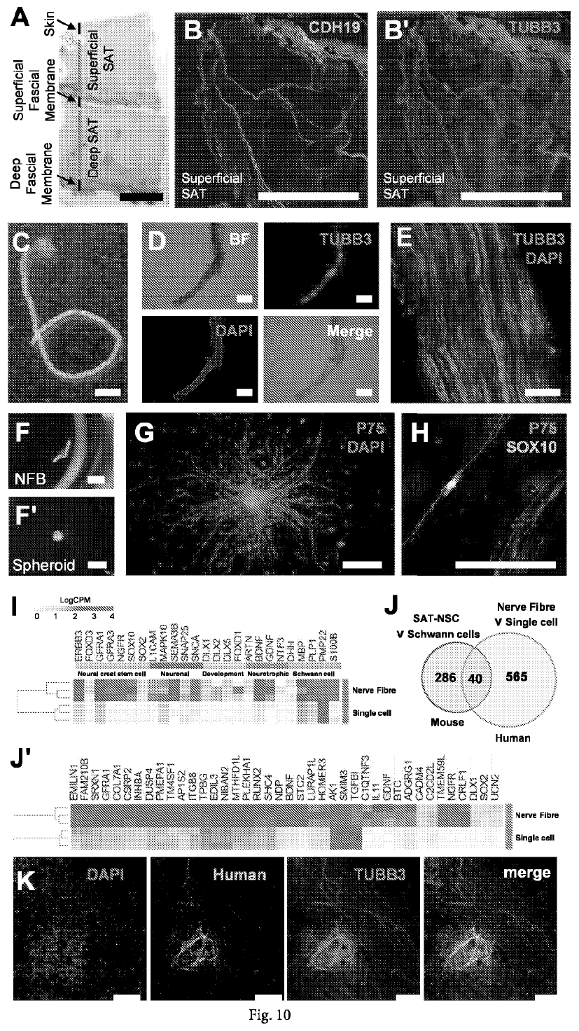

The data in this study indicates that the NFBs in mice contain the niche of NSCs in the SAT. Human adipose has both sympathetic and sensory innervation. Nevertheless, recent mapping of the human adipose tissue by single-cell RNA-sequencing failed to detect any glial cell populations (Vijay et al., 2020). Considering studies isolating cells from the SAT are predominantly filtered to obtain single cell suspensions, the cell composition of undigested NFBs was examined to determine whether it may contain human SAT-NSCs. Human abdominal SAT specimens (FIG. 10 Panel A) were observed to contain NFBs as indicated by CDH19 and TUBB3 immunoreactive fibres in cross sections of the superficial SAT (FIG. Panel B). Human SAT was minced, digested, and filtered through a 70 μm strainer. The counter-filtered material was examined under light dissection microscopy and NFBs were manually selected based on their characteristic striations and ‘frayed’ edges observed at the sites of NFB severance (FIG. 10 Panel C). Unlike in mice, high quantities of blood vessels and connective tissue were present in the counter-filtered SAT. Isolation of NFBs was confirmed by immunoreactivity for TUBB3 (FIG. 10 Panel D). Nucleated cells were observed juxtaposed to TUBB3 nerve fibres in wholemounts (FIG. 10 Panel E) of the human SAT NFB. Similar to mice, human SAT NFBs could be cultured in vitro in neuroproliferation medium (FIG. 10 Panel F), where they self-remodeled into free-floating spheroid structures, and this was observed for NFBs obtained from all subjects (n=3) (FIG. 10 Panel F′). In SAT processed by enzymatic and mechanical dissociation, 9/9 NFBs formed spheroids, in contrast to only 4/18 NFBs obtained from mechanical digestion alone. Spheroids generated from NFBs exhibited a 63.7 fold change (P<0.0001, n=3) in PLP1 expression compared to those derived from cells of filtered SAT cultured in the same medium (n=4), confirming the successful enrichment of SAT-NSCs from NFBs as observed in mice. Human SAT NFB-derived spheroids were subsequently cultured on a fibronectin coated surface to promote cell migration. Although spheroids were heterogenous, high numbers of cells morphologically similar to mouse SAT-NSCs were observed. These cells expressed the neural crest marker P75, which was identified as a specific marker for SAT-NSCs in mice (FIG. 10 Panel G), and colocalised with the expression of the NC, NSC, and glial cell marker, SOX10 (FIG. 10 Panel H). Low-input RNA-Seq was performed on SAT NFB-derived spheroids and spheroids generated from filtered single cell suspensions of adipose (not containing NFBs) obtained from the same donors and cultured in the same conditions. A total of 2421 DEGs (FDR 0.05) were identified between these cells (Data File S1). As shown in FIG. 10I, genes significantly upregulated in SAT NFB-derived spheroids included key neural crest stem cell markers such as NGFR (594 fold) and SOX10 (417 fold); neuronal marker SNAP25 (4.6 fold); neurotrophic factors, BDNF (68.8 fold) and GDNF (25.7 fold); and PLP1 (60.1 fold). To identify potential markers of SAT-NSCs, the top 605 upregulated genes (FDR 0.01) in this dataset were compared to our previous analysis of mouse SAT-NSCs and Schwann cells (FIG. 5 Panel B) and identified 40 shared upregulated genes in SAT-NSCs from both species (FIG. 10 Panel J). This included several markers such as NGFR (594 fold), TMEM59L (1226.5 fold), GFRA1 (9.3 fold) and ITGB8 (23.2 fold). Human SAT NFB-derived spheroids were transplanted ex vivo onto mouse colon, where the cells engrafted, migrated, and expressed TUBB3 after 7 days in culture, consistent with enteric neuronal differentiation (FIG. 10 Panel K). These findings confirm that human subcutaneous fat contains a similar population of SAT-NSCs as identified in the mouse. Importantly, these cells express similar markers, such as NGFR (P75) and PLP1, can be isolated from human SAT NFBs, and possess neurogenic potential.

### Example 8. Human SAT-NSCs Demonstrate Neuronal Function in the Colorectum

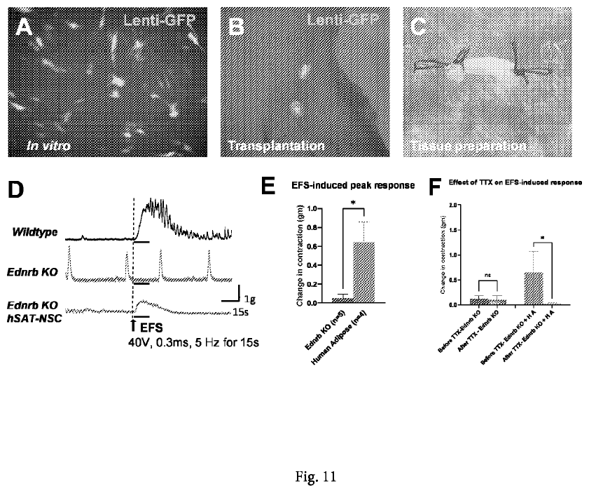

To examine the functional potential of human SAT-NSCs, transplantation experiments were conducted in the Ednrb null model of intestinal aganglionosis. Human SAT-NSCs were cultured on fibronectin to promote monolayer formation. This was performed to increase the surface area for lentiviral-delivery of GFP for cell tracing (FIG. 11 Panel A). Cells were washed with fresh media after 48 h, trypsinized, and then cultured in low-attachment conditions to reform neurospheres for 10 days. Neurospheres were transplanted to ex vivo preparations of the muscularis externa from the distal colon of Ednrb null mice and preparations were cultured for 6 days (FIG. 11 Panel B). To measure force contraction, muscle sheets were folded along the circumferential axis and tied at each end with nylon sutures to create a hairpin-like loop (FIG. 11 Panel C). These loops were hooked onto force transducers in a muscle strip myograph bath system (Model 820 MS; Danish Myo Technology, Aarhus, Denmark) to quantify smooth muscle contraction. Ednrb null tissues transplanted with human SAT-NSCs exhibited robust contractions in response to stimulation by EFS, as observed in wildtype tissues, but not untreated tissues from Ednrb null mice (FIG. 11 Panel D). Transplantation of human SAT-NSCs significantly increased contractile responses to EFS in tissues from aganglionic mice (FIG. 11 Panel E). Further, these contractions were inhibited in a TTX dependent manner indicating that they were neurally-mediated (FIG. 11 Panel F).

### Example 9. NSCs can be Isolated from the Mesenteric Fat of Mice

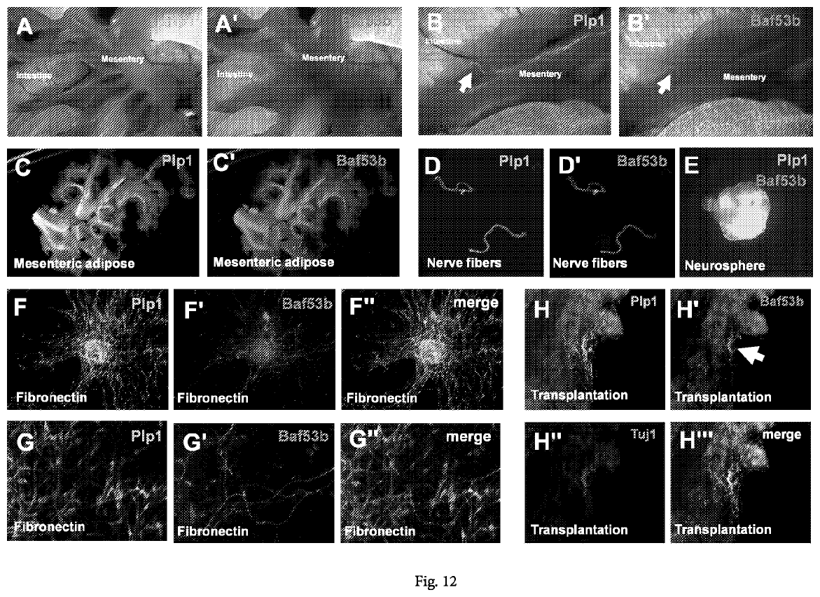

Our previous data provides evidence of NSC populations in the NFBs of SAT. Similar nerve fibers were also visualized in the visceral adipose tissue (VAT) of Plp1-GFP; Baf53b-tdT reporter mice whereby glia (Schwann cells) express GFP and neurons, including their nerve fiber projections, express tdT (FIG. 12 Panel A). Glia and neural projections are observed in close opposition with nerve fiber bundles running throughout the mesenteric adipose towards, and finally innervating, the small bowel (FIG. 12 Panel B). Mesenteric adipose tissue was harvested from these reporter mice (FIG. 12 Panel C). To isolate nerve fiber bundles, tissues where enzymatically dissociated in collagenase-dispase solution for 40 minutes and triturated to generate cell suspensions. These solutions were filtered through a porous membrane with a pore-size of 40 μm. The flow through was discarded and the remaining material was collected by inverting the filter and washing the membrane with cell culture media. Nerve fiber bundles identified by Plp1-GFP; Baf53b-tdT were observed in the counter-filtered material (FIG. 12 Panel D). Nerve fiber bundles cultured in neuroproliferation medium for 5 days had formed neurospheres and lost expression of tdT (FIG. 12 Panel E). Neurospheres cultured on fibronectin for 10 days in the presence of FBS exhibited cell migration and expression of Baf53b-tdT (FIG. 12 Panel F) which was accompanied by morphologically distinct elongated nerve fiber projections confirming the presence of neurons (FIG. 12 Panel G). Similarly, neurospheres transplanted to ex vivo preparations of the colonic muscularis migrated in the intestinal microenvironment and show evidence of neuronal differentiation by Baf53b-tdT and immunoreactivity for the neuronal marker Tuj1 (TUBB3) (FIG. 12 Panel H).

### Example 10. Isolation of Neurospheres from the NFBs of VAT from Human Specimens

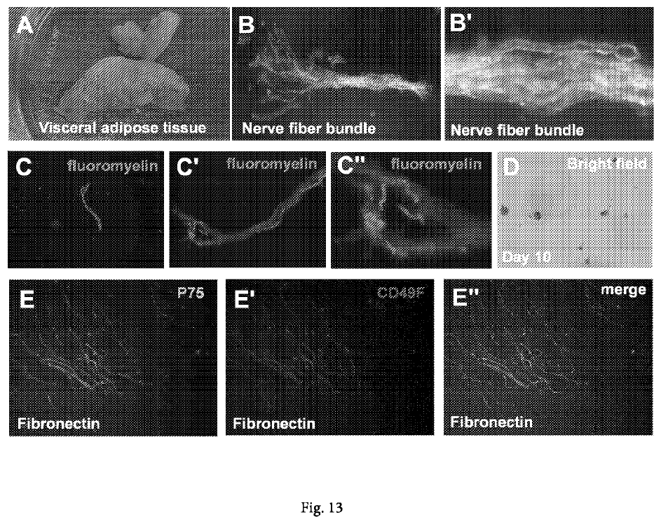

VAT specimens were collected from the omental fat depot during laparoscopic surgery (FIG. 13 Panel A). Tissues were minced and digested in 1 mL of collagenase XI (1 mg mL-1; Sigma Aldrich, St. Louis, Missouri) and dispase (250 μg mL-1; STEMCELL Technologies, Vancouver, Canada) solution per 0.2 grams of tissue and incubated at 37° C. for 4 hours to generate liquid cell solutions. The counter filtered material>70 μm was collected as described in mice and plated in a sterile petri dish. The counter filtered material was visually inspected and NFBs were identified by characteristic longitudinal striations, frayed edges, and the bead-like appearance of the myelinated nerve fibers (FIG. 13 Panel B). Similar to observations in specimens of SAT, isolated NFBs from the VAT generated neurospheres after culture in neuroproliferation medium in low-attachment conditions. NFBs were found to stain with the non-toxic dye fluoromyelin red (FIG. 13 Panel C) validating that the counter filtered material successfully enriches for NFBs. Culture of stained NFBs were still capable of generating neurospheres after 10 days (FIG. 13 Panel D) which provides a potential aid in the method of NFB isolation. Neurospheres migrated fibronectin-coated culture dishes in the presence of FBS and with cells expressing P75 and CD49F, similar to what as observed with SAT-NSCs (FIG. 13 Panel E-Panel E″).

### Example 11. Enrichment of NSC Populations in Adipose-Derived Neurospheres Using Counter Filtration

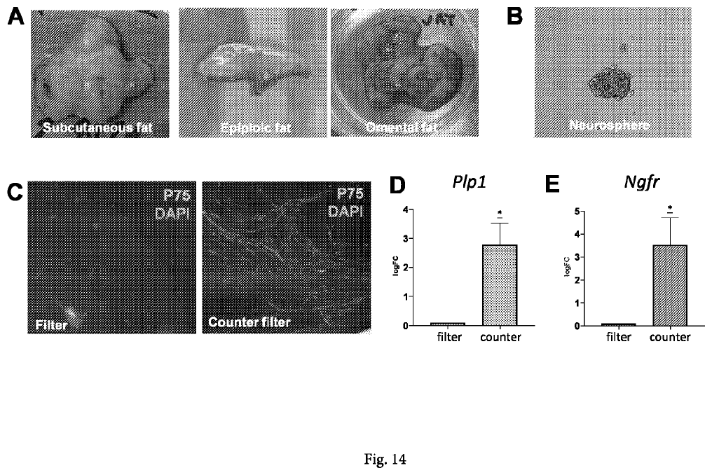

Specimens of SAT and VAT, including epiploic and omental adipose tissue were collected by laparoscopic surgical procedures (FIG. 14 Panel A). Tissues were processed as described in example 10 above. The counter filtered material containing NFBs and the filtered cells were immediately cultured in neuroproliferation medium in low-attachment conditions to generate spheroids which could be observed after 10 days (FIG. 14 Panel B). Spheroids generated from the filtered cell suspensions and counter filtered material were cultured on fibronectin-coated cell culture dishes in the presence of FBS for two weeks. Visualization of P75 expression confirmed the enrichment of NSCs from the counter filtered material (FIG. 14 Panel C). In further experiments, cells were trypsinized and allowed to reform spheroids in low-attachment conditions for two weeks. PCR analysis on spheroids generated from the filtered cells and the counter filtered material indicated 6.5 times the expression of Plp1 (FIG. 14 Panel D) and 11 times the expression of Ngfr (FIG. 14 Panel E) on average in neuropsheres from the counter filtered material compared to spheroids from filtered single cell suspensions.

### Example 12. Application of SAT-NSCs for Peripheral Nerve Injury

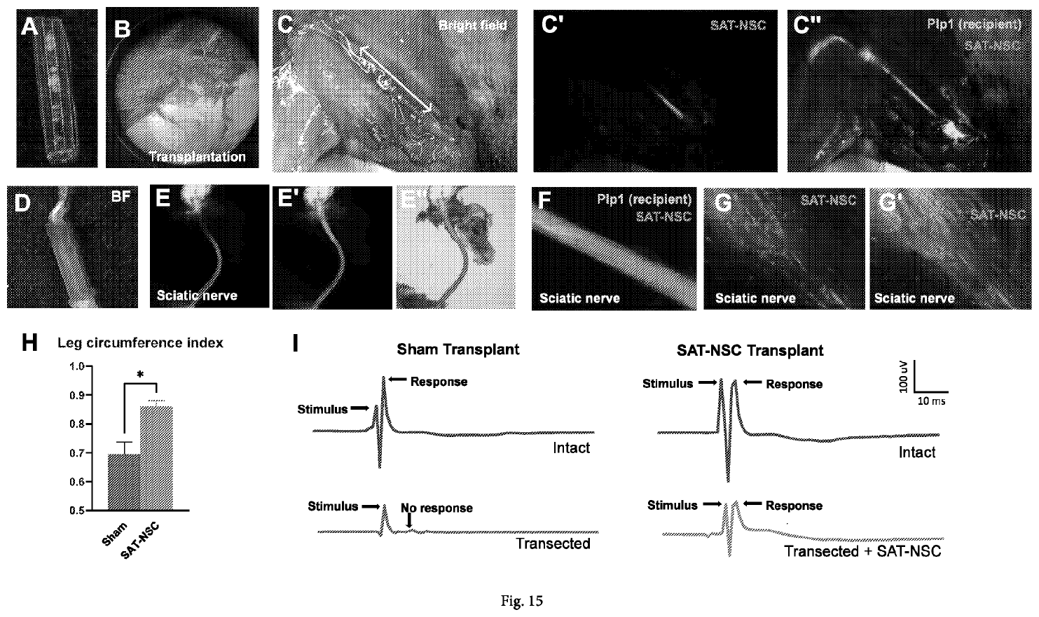

SAT-NSC enriched neurospheres were generated from Wnt1-tdT mice and transferred to the lumen of a 7 mm silicone tube acting as a conduit to apply cells for nerve gap repair (FIG. 15 Panel A). Plp1-GFP mice were anesthetized using isoflurane and the sciatic nerve was transected leaving a 5 mm nerve gap. The proximal and distal stumps of the sciatic nerve were sutured 1 mm into each end of the silicone conduit leaving a 5 mm gap containing SAT-NSCs or cell culture media as a sham control (FIG. 15 Panel B). After 4 weeks, mice were sacrificed, and the nerves were examined. Full reformation of the nerve as visualized by Plp1-GFP expression was only observed in SAT-NSC treated mice (FIG. 15 Panel C-Panel C″). The conduit along with the proximal and distal ends of the sciatic nerve was removed for further inspection of nerve regrowth (FIG. 15 Panel D). Survival of SAT-NSCs in and around the nerve was confirmed by tdT expression (FIG. 15 Panel E-Panel E″). Closer inspection of the sciatic nerve revealed successful integration of SAT-NSCs with the Plp1-GFP expressing Schwann cells of the recipient (FIG. 15 Panel F). SAT-NSCs were observed to form elongated structures from the proximal to distal axis of the nerve reminiscent of Schwann cell morphology (FIG. 15 Panel G-Panel G′). The leg circumference index was determined by measuring the leg circumference 10 mm above the ankle as a ratio between the leg with sciatic nerve transection and the opposite leg with an intact sciatic nerve. The leg circumference index was higher in SAT-NSC treated mice compared to those with sham treatments indicating reduced atrophy caused by loss of innervation and inactivity (FIG. 15 Panel H). Electromyography recordings were taken in the gastrocnemius in response to stimulation of the sciatic nerve (1V, 0.2 ms, 1 Hz), proximal to the implanted conduit (FIG. 15 Panel I). Recordings were performed on the unoperated hindleg with an intact sciatic nerve as an internal control and to demonstrate normal responses (gray traces). These show an initial positive deflection caused by sciatic nerve stimulation followed by a second deflection signifying electrical activity in muscle in response to the nerve stimulation. In mice with sciatic nerve transection and sham-treatment, no electrical activity in the gastrocnemius was observed after sciatic nerve stimulation. Conversely, mice with SAT-NSC treatments exhibited electrical activity in the gastrocnemius after sciatic nerve stimulation indicating the restoration of conductivity across the transected sciatic nerve.

### Example 13. Application of Adipose-Derived NSCs to Treat Disorders of the Central Nervous System (CNS)

Cells may also be used to treat disorders of the CNS. This can include conditions such as neurodegenerative diseases, neurocognitive decline, traumatic brain injury, ischemic brain injury and spinal cord injury. Transplantation of adipose-derived NSCs can be achieved by several delivery methods. This can include intracerebroventricular injection, intrathecal injection, intranasal delivery, and stereotactic brain injection directly to CNS tissues which may include, but are not limited to, the prefrontal and parietal cortices, hippocampus and cerebral cortex. Injection of adipose-derived NSCs may be performed using intact neurospheres, or single cell suspensions produced by digesting neurospheres in enzymes such as Accutase (Thermofisher).

### REFERENCES

All patents, patent applications and publications mentioned in this specification are herein incorporated by reference to the same extent as if each independent patent and publication was specifically and individually indicated to be incorporated by reference.
Boesmans, W., Martens, M. A., Weltens, N., Hao, M. M., Tack, J., Cirillo, C., & Vanden Berghe, P. (2013). Imaging neuron-glia interactions in the enteric nervous system. Frontiers in cellular neuroscience, 7, 183.
Chu, D. T., Nguyen Thi Phuong, T., Tien, N. L. B., Tran, D. K., Minh, L. B., Thanh, V. V., . . . Thi Nga, V. (2019). Adipose Tissue Stem Cells for Therapy: An Update on the Progress of Isolation, Culture, Storage, and Clinical Application. Journal of clinical medicine, 8 (7), 917. doi: 10.3390/jcm8070917
Diehl, H. J., Schaich, M., Budzinski, R. M., & Stoffel, W. (1986). Individual exons encode the integral membrane domains of human myelin proteolipid protein. Proceedings of the National Academy of Sciences, 83 (24), 9807-9811.
El-Nachef, W. N., & Bronner, M. E. (2020). &lt;em&gt; De novo&lt;/em&gt; enteric neurogenesis in post-embryonic zebrafish from Schwann cell precursors rather than resident cell types. Development, 147 (13), dev186619. doi: 10.1242/dev.186619
Furlan, A., & Adameyko, I. (2018). Schwann cell precursor: a neural crest cell in disguise? Developmental Biology, 444, S25-S35. doi: https://doi.org/10.1016/j.ydbio.2018.02.008
Johnson, D., Lanahan, A., Buck, C. R., Sehgal, A., Morgan, C., Mercer, E., . . . Chao, M. (1986). Expression and structure of the human NGF receptor. Cell, 47 (4), 545-554. doi: 10.1016/0092-8674 (86) 90619-7
Kameneva, P., Kastriti, M. E., & Adameyko, I. (2020). Neuronal lineages derived from the nerve-associated Schwann cell precursors. Cellular and Molecular Life Sciences, 1-17.
Krabbe, C., Zimmer, J., & Meyer, M. (2005). Neural transdifferentiation of mesenchymal stem cells—a critical review. Apmis, 113 (11-12), 831-844.
Li, W., Huang, L., Zeng, J., Lin, W., Li, K., Sun, J., . . . Xiang, A. P. (2018). Characterization and transplantation of enteric neural crest cells from human induced pluripotent stem cells. Molecular Psychiatry, 23 (3), 499-508. doi: 10.1038/mp.2016.191
O'Leary, N. A., Wright, M. W., Brister, J. R., Ciufo, S., Haddad, D., Mcveigh, R., . . . Pruitt, K. D. (2016). Reference sequence (RefSeq) database at NCBI: current status, taxonomic expansion, and functional annotation. Nucleic Acids Res, 44 (D1), D733-745. doi: 10.1093/nar/gkv1189
Ota, T., Suzuki, Y., Nishikawa, T., Otsuki, T., Sugiyama, T., Irie, R., . . . Sugano, S. (2004). Complete sequencing and characterization of 21,243 full-length human cDNAs. Nat Genet, 36 (1), 40-45. doi: 10.1038/ng1285
Peng, C., Lu, L., Li, Y., & Hu, J. (2019). Neurospheres Induced from Human Adipose-Derived Stem Cells as a New Source of Neural Progenitor Cells. Cell transplantation, 28 (1_suppl), 66S-75S. doi: 10.1177/0963689719888619
Phinney, D. G., & Prockop, D. J. (2007). Concise review: mesenchymal stem/multipotent stromal cells: the state of transdifferentiation and modes of tissue repair—current views. STEM CELLS, 25 (11), 2896-2902.
Pusch, C., Hustert, E., Pfeifer, D., Südbeck, P., Kist, R., Roe, B., . . . Scherer, G. (1998). The SOX10/Sox10 gene from human and mouse: sequence, expression, and transactivation by the encoded HMG domain transcription factor. Hum Genet, 103 (2), 115-123. doi: 10.1007/s004390050793
Rajbhandari, P., Arneson, D., Hart, S. K., Ahn, I. S., Diamante, G., Santos, L. C., . . . Vergnes, L. (2019). Single cell analysis reveals immune cell-adipocyte crosstalk regulating the transcription of thermogenic adipocytes. Elife, 8, e49501.
Sowa, Y., Imura, T., Numajiri, T., Takeda, K., Mabuchi, Y., Matsuzaki, Y., & Nishino, K. (2013). Adipose stromal cells contain phenotypically distinct adipogenic progenitors derived from neural crest. PLoS One, 8 (12), e84206.
Uesaka, T., Nagashimada, M., & Enomoto, H. (2015). Neuronal Differentiation in Schwann Cell Lineage Underlies Postnatal Neurogenesis in the Enteric Nervous System. The Journal of Neuroscience, 35 (27), 9879. doi: 10.1523/JNEUROSCI.1239-15.2015
Vijay, J., Gauthier, M. F., Biswell, R. L., Louiselle, D. A., Johnston, J. J., Cheung, W. A., . . . Gibson, M. (2020). Single-cell analysis of human adipose tissue identifies depot- and disease-specific cell types. Nature Metabolism, 2 (1), 97-109.
Zuk, P. A., Zhu, M., Ashjian, P., De Ugarte, D. A., Huang, J. I., Mizuno, H., . . . Hedrick, M. H. (2002). Human adipose tissue is a source of multipotent stem cells. Molecular Biology of the Cell, 13 (12), 4279-4295.

What is claimed is:

1. A method of producing human neuronal progenitor cells from adipose tissue, said method comprising the steps of:
a) dissociating the adipose tissue to collect nerve fibre bundles (NFBs); and
b) culturing NFBs; thereby producing human neuronal progenitor cells from adipose tissue.

2. The method of claim 1, wherein step b) is conducted until spheroids are formed.

3. The method of claim 1 or 2, wherein the NFBs are collected by counter-filtration.

4. The method of any one of claims 1-3, wherein the nerve NFBs are visualized using a dye.

5. The method of claim 4, wherein the dye is fluoromyelin red.

6. The method of any one of claims 2-5, wherein the method further comprises the step of expanding the spheroids in human neural proliferation medium.

7. The method of any one of claims 1-5, wherein the NFBs are cultured on a fibronectin coated substrate in the presence of Fetal Bovine Serum.

8. The method of claim 1, wherein step a) comprises:
i) digesting the adipose tissue; and
ii) filtering the digested adipose tissue to separate lipids from NFBs.

9. The method of claim 8, wherein step i) is conducted by contacting the adipose tissue with one or more enzymes.

10. The method of claim 9, wherein the enzymes comprise a dispase and a collagenase.

11. The method of any one of claims 1-10, wherein the adipose tissue is subcutaneous adipose tissue (SAT).

12. The method of any one of claims 1-10, wherein the adipose tissue is visceral adipose tissue (VAT).

13. The method of any one of claims 1-12, wherein the human neuronal progenitor cells express Plp1, P75 and SOX10 or express P75 and CD49f.

14. A human neuronal progenitor cell or a population of cells comprising a human neuronal progenitor cell obtained from the method of any one of claims 1-13, wherein said human neuronal progenitor cell expresses Plp1, P75 and SOX10.

15. A human neuronal progenitor cell or a population of cells comprising a human neuronal progenitor cell obtained from the method of any one of claims 1-13, wherein said human neuronal progenitor cell expresses P75 and CD49f.

16. The human neuronal progenitor cell of claim 14 or 15, wherein the progenitor cell does not express Zic1 or does not express a significant amount of Zic1.

17. A method of producing neuronal function in the colorectum or stomach of a subject in need thereof, said method comprising contacting the smooth muscle wall of the colorectum or stomach with the human neuronal progenitor cell or a population of cells comprising a human neuronal progenitor cell of claim 14 or 15, wherein said human neuronal progenitor cell engrafts into the colorectum or stomach and produces neuronal function.

18. The method of claim 17, wherein the subject suffers from Hirschsprung disease.

19. The method of claim 17, wherein the subject suffers from gastroparesis.

20. A method of producing neuronal function in the nervous system of a subject in need thereof, said method comprising contacting nerve tissue with the human neuronal progenitor cell or a population of cells comprising a human neuronal progenitor cell of claim 14 or 15, wherein said human neuronal progenitor cell produces neuronal function in the nerve tissue.

21. The method of claim 20, wherein the nerve tissue is damaged.

22. The method of claim 20 or 21, wherein the nerve tissue is in the peripheral nervous system.

23. The method of claim 20 or 21, wherein the nerve tissue is in the central nervous system.

24. The method of claim 20 or 21, wherein the nerve tissue is in the enteric nervous system.
